SEQUENCE LISTING (300 sequences)

SEQ ID NO: 1 - protein, 8 aa, Mus musculus
SPSYVYHQ

SEQ ID NO: 2 - protein, 11 aa, Mus musculus
TQQYHQLKTIG

SEQ ID NO: 3 - protein, 9 aa, Mus musculus
SMAKLRERL

SEQ ID NO: 4 - protein, 9 aa, Mus musculus
SGPPYYEGV

SEQ ID NO: 5 - protein, 9 aa, Mus musculus
VLTQQYHQL

SEQ ID NO: 6 - protein, 11 aa, Mus musculus
LVLTQQYHQLK

SEQ ID NO: 7 - protein, 10 aa, Mus musculus
VLTQQYHQLK

SEQ ID NO: 8 - protein, 9 aa, Mus musculus
WFTTLISTI

SEQ ID NO: 9 - protein, 8 aa, Mus musculus
LIILLLIL

SEQ ID NO: 10 - protein, 8 aa, Mus musculus
HGPSYWGL

SEQ ID NO: 11 - protein, 9 aa, Mus musculus
DYITVSNNL

SEQ ID NO: 12 - protein, 8 aa, Mus musculus
PSYVYHQF

SEQ ID NO: 13 - protein, 8 aa, Mus musculus
CFYADHTG

SEQ ID NO: 14 - protein, 9 aa, Mus musculus
AAPTGTTWA

SEQ ID NO: 15 - protein, 13 aa, Mus musculus
VLTQQYHQLKTIG

SEQ ID NO: 16 - protein, 9 aa, Mus musculus
LGGVNPVAL

SEQ ID NO: 17 - protein, 10 aa, Mus musculus
CCFYADHTGL

SEQ ID NO: 18 - protein, 9 aa, Mus musculus
CFYADHTGL

SEQ ID NO: 19 - protein, 8 aa, Mus musculus
FYADHTGL

SEQ ID NO: 20 - protein, 9 aa, Mus musculus
SPSYVYHQF

SEQ ID NO: 21 - protein, 9 aa, Mus musculus
TSAQRAELI

SEQ ID NO: 22 - protein, 11 aa, Mus musculus
HPTVPNPYNLL

SEQ ID NO: 23 - protein, 11 aa, Mus musculus
LPQGFKNSPTL

SEQ ID NO: 24 - protein, 9 aa, Mus musculus
WYTDGSSFL

SEQ ID NO: 25 - protein, 10 aa, Mus musculus
HPTVPNPYNL

SEQ ID NO: 26 - protein, 8 aa, Mus musculus
YQKMKALL

SEQ ID NO: 27 - protein, 9 aa, Mus musculus
YPLLGRDLL

SEQ ID NO: 28 - protein, 10 aa, Mus musculus
AYQEIKQALL

SEQ ID NO: 29 - protein, 10 aa, Mus musculus
HPTSQPLFAF

SEQ ID NO: 30 - protein, 8 aa, Mus musculus
YQEIKQAL

SEQ ID NO: 31 - protein, 9 aa, Mus musculus
GYQKMKALL

SEQ ID NO: 32 - protein, 8 aa, Mus musculus
VGPKGQPL

SEQ ID NO: 33 - protein, 9 aa, Mus musculus
AYQEIKQAL

SEQ ID NO: 34 - protein, 9 aa, Mus musculus
PTVPNPYNL

SEQ ID NO: 35 - protein, 8 aa, Mus musculus
TVPNPYNL

SEQ ID NO: 36 - protein, 9 aa, Mus musculus
KPSPPPSEF

SEQ ID NO: 37 - protein, 13 aa, Mus musculus
GLQNAGRSPTNLA

SEQ ID NO: 38 - protein, 9 aa, Mus musculus
QVLSDNGGP

SEQ ID NO: 39 - protein, 11 aa, Mus musculus
PLRLGGNGQLQ

SEQ ID NO: 40 - protein, 8 aa, Mus musculus
SPDRFGLF

SEQ ID NO: 41 - protein, 13 aa, Mus musculus
PPPYGGQGPSSSD

SEQ ID NO: 42 - protein, 8 aa, Mus musculus
PQDGTFNL

SEQ ID NO: 43 - protein, 9 aa, Mus musculus
TPYDPEDPG

SEQ ID NO: 44 - protein, 9 aa, Mus musculus
WPQDGTFNL

SEQ ID NO: 45 - protein, 12 aa, Mus musculus
PPAADSTTSRAF

SEQ ID NO: 46 - protein, 10 aa, Mus musculus
GEEKQRVLLE

SEQ ID NO: 47 - protein, 13 aa, Mus musculus
TNLAKVKGITQGP

SEQ ID NO: 48 - protein, 10 aa, Mus musculus
SAPDIGRKLE

SEQ ID NO: 49 - protein, 8 aa, Mus musculus
FYADHTGI

SEQ ID NO: 50 - protein, 9 aa, Mus musculus
SRTEADYYL

SEQ ID NO: 51 - protein, 9 aa, Mus musculus
SGPPYYKGI

SEQ ID NO: 52 - protein, 10 aa, Mus musculus
CCFYADHTGI

SEQ ID NO: 53 - protein, 9 aa, Mus musculus
CFYADHTGI

SEQ ID NO: 54 - protein, 9 aa, Mus musculus
KYFPANNKI

SEQ ID NO: 55 - protein, 9 aa, Mus musculus
QGPQIYGAL

SEQ ID NO: 56 - protein, 9 aa, Mus musculus
YYIPGLKGI

SEQ ID NO: 57 - protein, 9 aa, Mus musculus
DYLANIHHL

SEQ ID NO: 58 - protein, 10 aa, Mus musculus
HPNSQPLFAF

SEQ ID NO: 59 - protein, 9 aa, Mus musculus
TYVAGDTQV

SEQ ID NO: 60 - protein, 9 aa, Mus musculus
WPAEGTFYL

SEQ ID NO: 61 - protein, 9 aa, Mus musculus
FYLPTIRAV

SEQ ID NO: 62 - protein, 8 aa, Mus musculus
PAEGTFYL

SEQ ID NO: 63 - protein, 9 aa, Mus musculus
SYVPGGFDL

SEQ ID NO: 64 - protein, 9 aa, Mus musculus
SGPPYYEGI

SEQ ID NO: 65 - protein, 8 aa, Mus musculus
VGPNKVLI

SEQ ID NO: 66 - protein, 9 aa, Mus musculus
RSPVYITHV

SEQ ID NO: 67 - protein, 9 aa, Mus musculus
SSFGKLQYL

SEQ ID NO: 68 - protein, 8 aa, Mus musculus
QSVVFPQI

SEQ ID NO: 69 - protein, 9 aa, Mus musculus
SPVEAARNF

SEQ ID NO: 70 - protein, 11 aa, Mus musculus
KYGSQNKYTGL

SEQ ID NO: 71 - protein, 9 aa, Mus musculus
GYLISNQQV

SEQ ID NO: 72 - protein, 8 aa, Mus musculus
AIPKDFNP

SEQ ID NO: 73 - protein, 9 aa, Mus musculus
SYQKMRALL

SEQ ID NO: 74 - protein, 9 aa, Mus musculus
LPDSGGPLI

SEQ ID NO: 75 - protein, 9 aa, Mus musculus
NGPAYTSQI

SEQ ID NO: 76 - protein, 9 aa, Mus musculus
GYQPGKVLA

SEQ ID NO: 77 - protein, 11 aa, Mus musculus
KYGTQNKYTGL

SEQ ID NO: 78 - protein, 8 aa, Mus musculus
APVEYLQI

SEQ ID NO: 79 - protein, 9 aa, Mus musculus
SPVEAAKNF

SEQ ID NO: 80 - protein, 9 aa, Mus musculus
AYTPGTTAV

SEQ ID NO: 81 - protein, 9 aa, Mus musculus
GPQESFSDF

SEQ ID NO: 82 - protein, 10 aa, Mus musculus
AYAQISSTAI

SEQ ID NO: 83 - protein, 9 aa, Mus musculus
QGPPYAESL

SEQ ID NO: 84 - protein, 9 aa, Mus musculus
LGPPYYEGI

SEQ ID NO: 85 - protein, 10 aa, Mus musculus
ECCYADHTGI

SEQ ID NO: 86 - protein, 10 aa, Mus musculus
AYNTGLTPCV

SEQ ID NO: 87 - protein, 11 aa, Mus musculus
DCCFYADHTGI

SEQ ID NO: 88 - protein, 9 aa, Mus musculus
VPLIITPAI

SEQ ID NO: 89 - protein, 8 aa, Mus musculus
KPSSSWDL

SEQ ID NO: 90 - protein, 9 aa, Mus musculus
YLASPAGTI

SEQ ID NO: 91 - protein, 9 aa, Mus musculus
LGPSTSTQI

SEQ ID NO: 92 - protein, 13 aa, Mus musculus
LTEDPPAVRSTTF

SEQ ID NO: 93 - protein, 10 aa, Mus musculus
KGPANIFNKI

SEQ ID NO: 94 - protein, 9 aa, Mus musculus
LYLNIIKTI

SEQ ID NO: 95 - protein, 9 aa, Mus musculus
TPGDRIAQL

SEQ ID NO: 96 - protein, 9 aa, Mus musculus
LVDSGAQVS

SEQ ID NO: 97 - protein, 8 aa, Mus musculus
FYADHTGL

SEQ ID NO: 98 - protein, 9 aa, Mus musculus
SMAKLRERL

SEQ ID NO: 99 - protein, 9 aa, Mus musculus
QYHQLKTIG

SEQ ID NO: 100 - protein, 9 aa, Mus musculus
VLTQQYHQL

SEQ ID NO: 101 - protein, 8 aa, Mus musculus
SPHQVFNL

SEQ ID NO: 102 - protein, 9 aa, Mus musculus
SPSYVYHQF

SEQ ID NO: 103 - protein, 8 aa, Mus musculus
ISTTILDL

SEQ ID NO: 104 - protein, 8 aa, Mus musculus
MAKLRERL

SEQ ID NO: 105 - protein, 8 aa, Mus musculus
CFYADHTG

SEQ ID NO: 106 - protein, 9 aa, Mus musculus
CFYADHTGL

SEQ ID NO: 107 - protein, 8 aa, Mus musculus
PSYVYHQF

SEQ ID NO: 108 - protein, 12 aa, Mus musculus
IAAGVGTGTTAL

SEQ ID NO: 109 - protein, 8 aa, Mus musculus
HGPSYWGL

SEQ ID NO: 110 - protein, 8 aa, Mus musculus
GPCILNRL

SEQ ID NO: 111 - protein, 9 aa, Mus musculus
FGPCILNRL

SEQ ID NO: 112 - protein, 8 aa, Mus musculus
FGPCILNR

SEQ ID NO: 113 - protein, 11 aa, Mus musculus
LGPWRRPVAYL

SEQ ID NO: 114 - protein, 9 aa, Mus musculus
GYQKMKALL

SEQ ID NO: 115 - protein, 9 aa, Mus musculus
PTVPNPYNL

SEQ ID NO: 116 - protein, 8 aa, Mus musculus
TVPNPYNL

SEQ ID NO: 117 - protein, 9 aa, Mus musculus
TSAQRAELI

SEQ ID NO: 118 - protein, 11 aa, Mus musculus
HPTVPNPYNLL

SEQ ID NO: 119 - protein, 10 aa, Mus musculus
HPTVPNPYNL

SEQ ID NO: 120 - protein, 9 aa, Mus musculus
TVPNPYNLL

SEQ ID NO: 121 - protein, 8 aa, Mus musculus
VGPKGQPL

SEQ ID NO: 122 - protein, 9 aa, Mus musculus
VPCQSPWNT

SEQ ID NO: 123 - protein, 8 aa, Mus musculus
GYQKMKAL

SEQ ID NO: 124 - protein, 9 aa, Mus musculus
WYTDGSSFL

SEQ ID NO: 125 - protein, 9 aa, Mus musculus
YPLLGRDLL

SEQ ID NO: 126 - protein, 9 aa, Mus musculus
AYQEIKQAL

SEQ ID NO: 127 - protein, 8 aa, Mus musculus
YQEIKQAL

SEQ ID NO: 128 - protein, 10 aa, Mus musculus
AWAETGGMGL

SEQ ID NO: 129 - protein, 8 aa, Mus musculus
YQKMKALL

SEQ ID NO: 130 - protein, 10 aa, Mus musculus
AYQEIKQALL

SEQ ID NO: 131 - protein, 9 aa, Mus musculus
LYPALTPSI

SEQ ID NO: 132 - protein, 9 aa, Mus musculus
TPYDPEDPG

SEQ ID NO: 133 - protein, 8 aa, Mus musculus
PQDGTFNL

SEQ ID NO: 134 - protein, 11 aa, Mus musculus
PAADSTTSRAF

SEQ ID NO: 135 - protein, 9 aa, Mus musculus
KPSPPPSEF

SEQ ID NO: 136 - protein, 8 aa, Mus musculus
SPDRFGLF

SEQ ID NO: 137 - protein, 8 aa, Mus musculus
WPFSSSDL

SEQ ID NO: 138 - protein, 8 aa, Mus musculus
WPQDGTFN

SEQ ID NO: 139 - protein, 9 aa, Mus musculus
WPQDGTFNL

SEQ ID NO: 140 - protein, 9 aa, Mus musculus
EDPGKLTAL

SEQ ID NO: 141 - protein, 12 aa, Mus musculus
PPAADSTTSRAF

SEQ ID NO: 142 - protein, 8 aa, Mus musculus
FGPCIINR

SEQ ID NO: 143 - protein, 9 aa, Mus musculus
CFYADHTGI

SEQ ID NO: 144 - protein, 8 aa, Mus musculus
FYADHTGI

SEQ ID NO: 145 - protein, 9 aa, Mus musculus
SGPPYYKGI

SEQ ID NO: 146 - protein, 8 aa, Mus musculus
PPIKDDLI

SEQ ID NO: 147 - protein, 8 aa, Mus musculus
YFPANNKI

SEQ ID NO: 148 - protein, 9 aa, Mus musculus
KYFPANNKI

SEQ ID NO: 149 - protein, 9 aa, Mus musculus
QGPQIYGAL

SEQ ID NO: 150 - protein, 9 aa, Mus musculus
WTAVLPYAL

SEQ ID NO: 151 - protein, 9 aa, Mus musculus
DYLANIHHL

SEQ ID NO: 152 - protein, 9 aa, Mus musculus
WPAEGTFYL

SEQ ID NO: 153 - protein, 9 aa, Mus musculus
FYLPTIRAV

SEQ ID NO: 154 - protein, 9 aa, Mus musculus
LPDSGGPLI

SEQ ID NO: 155 - protein, 8 aa, Mus musculus
NPVITDQL

SEQ ID NO: 156 - protein, 9 aa, Mus musculus
SPVEAARNF

SEQ ID NO: 157 - protein, 9 aa, Mus musculus
AYVANGKVV

SEQ ID NO: 158 - protein, 11 aa, Mus musculus
GYQSCPTISSV

SEQ ID NO: 159 - protein, 8 aa, Mus musculus
APVKYLQI

SEQ ID NO: 160 - protein, 9 aa, Mus musculus
HAPVKYLQI

SEQ ID NO: 161 - protein, 8 aa, Mus musculus
AYAQISST

SEQ ID NO: 162 - protein, 10 aa, Mus musculus
QGPQRYGNRF

SEQ ID NO: 163 - protein, 8 aa, Mus musculus
YQKMRALL

SEQ ID NO: 164 - protein, 9 aa, Mus musculus
SYQKMRALL

SEQ ID NO: 165 - protein, 9 aa, Mus musculus
NGPAYTSQI

SEQ ID NO: 166 - protein, 8 aa, Mus musculus
VGPNKVLI

SEQ ID NO: 167 - protein, 9 aa, Mus musculus
WYLSDHTDL

SEQ ID NO: 168 - protein, 9 aa, Mus musculus
PGPHRSQSL

SEQ ID NO: 169 - protein, 8 aa, Mus musculus
AIPKDFNP

SEQ ID NO: 170 - protein, 9 aa, Mus musculus
LGPPYYEGI

SEQ ID NO: 171 - protein, 8 aa, Mus musculus
LGPPYYEG

SEQ ID NO: 172 - protein, 9 aa, Mus musculus
GYQPGKVLA

SEQ ID NO: 173 - protein, 8 aa, Mus musculus
METLISHL

SEQ ID NO: 174 - protein, 9 aa, Mus musculus
LGPTVPYTL

SEQ ID NO: 175 - protein, 10 aa, Mus musculus
MGPHKITKLL

SEQ ID NO: 176 - protein, 9 aa, Mus musculus
AYQKIKQAL

SEQ ID NO: 177 - protein, 9 aa, Mus musculus
LGPSTSTQI

SEQ ID NO: 178 - protein, 10 aa, Mus musculus
PAAAPPQGPI

SEQ ID NO: 179 - protein, 11 aa, Mus musculus
PAAAPPQGPIA

SEQ ID NO: 180 - protein, 9 aa, Mus musculus
RPKESPFEF

SEQ ID NO: 181 - protein, 10 aa, Mus musculus
AYVPLTTTAL

SEQ ID NO: 182 - protein, 8 aa, Mus musculus
AYVPLTTT

SEQ ID NO: 183 - protein, 9 aa, Mus musculus
AYVPLTTTA

SEQ ID NO: 184 - protein, 9 aa, Mus musculus
AYTPGTTAV

SEQ ID NO: 185 - protein, 9 aa, Mus musculus
LYLNIIKTI

SEQ ID NO: 186 - protein, 10 aa, Mus musculus
PAAAPPQGPL

SEQ ID NO: 187 - protein, 11 aa, Mus musculus
PAAAPPQGPLA

SEQ ID NO: 188 - protein, 11 aa, Mus musculus
LTFGPCILNRL

SEQ ID NO: 189 - protein, 9 aa, Mus musculus
LSLPRSKLI

SEQ ID NO: 190 - protein, 10 aa, Mus musculus
CCFYSDHTGI

SEQ ID NO: 191 - protein, 8 aa, Mus musculus
SVSRTEAI

SEQ ID NO: 192 - protein, 9 aa, Mus musculus
VLTQQYHQL

SEQ ID NO: 193 - protein, 10 aa, Mus musculus
CCFYADHTGL

SEQ ID NO: 194 - protein, 9 aa, Mus musculus
AAPTGTTWA

SEQ ID NO: 195 - protein, 9 aa, Mus musculus
LIILLLILL

SEQ ID NO: 196 - protein, 8 aa, Mus musculus
ATQQFQQL

SEQ ID NO: 197 - protein, 8 aa, Mus musculus
FYADHTGL

SEQ ID NO: 198 - protein, 9 aa, Mus musculus
SPSYVYHQF

SEQ ID NO: 199 - protein, 8 aa, Mus musculus
FTTLISTI

SEQ ID NO: 200 - protein, 9 aa, Mus musculus
FGPCILNRL

SEQ ID NO: 201 - protein, 9 aa, Mus musculus
LGGVNPVAL

SEQ ID NO: 202 - protein, 8 aa, Mus musculus
KSPWFTTL

SEQ ID NO: 203 - protein, 8 aa, Mus musculus
PSYVYHQF

SEQ ID NO: 204 - protein, 9 aa, Mus musculus
CFYADHTGL

SEQ ID NO: 205 - protein, 8 aa, Mus musculus
VGPKGQPL

SEQ ID NO: 206 - protein, 8 aa, Mus musculus
TVPNPYNL

SEQ ID NO: 207 - protein, 10 aa, Mus musculus
AYQEIKQALL

SEQ ID NO: 208 - protein, 9 aa, Mus musculus
GYQKMKALL

SEQ ID NO: 209 - protein, 9 aa, Mus musculus
AYQEIKQAL

SEQ ID NO: 210 - protein, 8 aa, Mus musculus
YQEIKQAL

SEQ ID NO: 211 - protein, 10 aa, Mus musculus
HPTSQPLFAF

SEQ ID NO: 212 - protein, 8 aa, Mus musculus
YQKMKALL

SEQ ID NO: 213 - protein, 11 aa, Mus musculus
LPQGFKNSPTL

SEQ ID NO: 214 - protein, 9 aa, Mus musculus
WYTDGSSFL

SEQ ID NO: 215 - protein, 9 aa, Mus musculus
TSAQRAELI

SEQ ID NO: 216 - protein, 11 aa, Mus musculus
HPTVPNPYNLL

SEQ ID NO: 217 - protein, 10 aa, Mus musculus
HPTVPNPYNL

SEQ ID NO: 218 - protein, 9 aa, Mus musculus
YPLLGRDLL

SEQ ID NO: 219 - protein, 12 aa, Mus musculus
PPAADSTTSRAF

SEQ ID NO: 220 - protein, 9 aa, Mus musculus
TPYDPEDPG

SEQ ID NO: 221 - protein, 9 aa, Mus musculus
KPSPPPSEF

SEQ ID NO: 222 - protein, 9 aa, Mus musculus
WPQDGTFNL

SEQ ID NO: 223 - protein, 8 aa, Mus musculus
WPFSSSDL

SEQ ID NO: 224 - protein, 8 aa, Mus musculus
PQDGTFNL

SEQ ID NO: 225 - protein, 10 aa, Mus musculus
CCFYADHTGI

SEQ ID NO: 226 - protein, 9 aa, Mus musculus
SGPPYYKGI

SEQ ID NO: 227 - protein, 8 aa, Mus musculus
FYADHTGI

SEQ ID NO: 228 - protein, 9 aa, Mus musculus
CFYADHTGI

SEQ ID NO: 229 - protein, 9 aa, Mus musculus
KYFPANNKI

SEQ ID NO: 230 - protein, 9 aa, Mus musculus
QGPQIYGAL

SEQ ID NO: 231 - protein, 9 aa, Mus musculus
TYVAGDTQV

SEQ ID NO: 232 - protein, 9 aa, Mus musculus
DYLANIHHL

SEQ ID NO: 233 - protein, 9 aa, Mus musculus
WTAVLPYAL

SEQ ID NO: 234 - protein, 9 aa, Mus musculus
YYIPGLKGI

SEQ ID NO: 235 - protein, 9 aa, Mus musculus
FYLPTIRAV

SEQ ID NO: 236 - protein, 8 aa, Mus musculus
PAEGTFYL

SEQ ID NO: 237 - protein, 9 aa, Mus musculus
WPAEGTFYL

SEQ ID NO: 238 - protein, 9 aa, Mus musculus
LPDSGGPLI

SEQ ID NO: 239 - protein, 9 aa, Mus musculus
SGPPYYEGI

SEQ ID NO: 240 - protein, 9 aa, Mus musculus
IPAGGAPLL

SEQ ID NO: 241 - protein, 8 aa, Mus musculus
APVKYLQI

SEQ ID NO: 242 - protein, 9 aa, Mus musculus
GPQESFSDF

SEQ ID NO: 243 - protein, 10 aa, Mus musculus
KYLQIKEIAE

SEQ ID NO: 244 - protein, 11 aa, Mus musculus
KYGTQNKYTGL

SEQ ID NO: 245 - protein, 9 aa, Mus musculus
SYQKMRALL

SEQ ID NO: 246 - protein, 12 aa, Mus musculus
FHNNFHVTAETL

SEQ ID NO: 247 - protein, 9 aa, Mus musculus
RSPVYITHV

SEQ ID NO: 248 - protein, 9 aa, Mus musculus
SPVEAARNF

SEQ ID NO: 249 - protein, 9 aa, Mus musculus
AYVANGKVV

SEQ ID NO: 250 - protein, 9 aa, Mus musculus
NGPAYTSQI

SEQ ID NO: 251 - protein, 8 aa, Mus musculus
QSVVFPQI

SEQ ID NO: 252 - protein, 9 aa, Mus musculus
SPVEAAKNF

SEQ ID NO: 253 - protein, 10 aa, Mus musculus
KYLQIKELAE

SEQ ID NO: 254 - protein, 9 aa, Mus musculus
QGPPYAESL

SEQ ID NO: 255 - protein, 9 aa, Mus musculus
GYQPGKVLA

SEQ ID NO: 256 - protein, 9 aa, Mus musculus
LGPPYYEGI

SEQ ID NO: 257 - protein, 10 aa, Mus musculus
ECCYADHTGI

SEQ ID NO: 258 - protein, 10 aa, Mus musculus
WCFYADHTGI

SEQ ID NO: 259 - protein, 10 aa, Mus musculus
NPTCGGKVDF

SEQ ID NO: 260 - protein, 9 aa, Mus musculus
KPGVVAHAF

SEQ ID NO: 261 - protein, 11 aa, Mus musculus
DCCFYADHTGI

SEQ ID NO: 262 - protein, 9 aa, Mus musculus
AYVPLTTTA

SEQ ID NO: 263 - protein, 10 aa, Mus musculus
AYVPLTTTAL

SEQ ID NO: 264 - protein, 10 aa, Mus musculus
AYNTGLTPCV

SEQ ID NO: 265 - protein, 9 aa, Mus musculus
LYLNIIKTI

SEQ ID NO: 266 - protein, 11 aa, Mus musculus
LTFGPCILNRL

SEQ ID NO: 267 - protein, 10 aa, Mus musculus
SCFYADHTGI

SEQ ID NO: 268 - protein, 9 aa, Mus musculus
AALQNPLTL

SEQ ID NO: 269 - protein, 9 aa, Mus musculus
LGPSTSTQI

SEQ ID NO: 270 - protein, 9 aa, Mus musculus
LGPPYYDGI

SEQ ID NO: 271 - protein, 27 aa, Mus musculus
IYEQATKECRAAIAPKKEQRLTRLAQG

SEQ ID NO: 272 - protein, 27 aa, Mus musculus
TYGIPAHNRQTLQSTELQKSQQEWISV

SEQ ID NO: 273 - protein, 27 aa, Mus musculus
MSIQRVPVEPIPSLPPGTMGLILGRGS

SEQ ID NO: 274 - protein, 27 aa, Mus musculus
AYQKIKQALLTAPALRLPDLTKPFELF

SEQ ID NO: 275 - protein, 27 aa, Mus musculus
PQGSRLQGPPYAESLPCVVRQPCAERQ

SEQ ID NO: 276 - protein, 27 aa, Mus musculus
NSPSLQAHLQALQAVQREVWKPLAAAY

SEQ ID NO: 277 - protein, 27 aa, Mus musculus
PQVLGTDNGPAFVSQVSQSVAKLLGID

SEQ ID NO: 278 - protein, 27 aa, Mus musculus
FLYSKLPSKLIQSTSSTSPRSLTSEGL

SEQ ID NO: 279 - protein, 27 aa, Mus musculus
PRSQGPQRYGNRFVRTQEAAREATQED

SEQ ID NO: 280 - protein, 27 aa, Mus musculus
PNSSKLTVAVAQSDAAGKSGPSAPPKI

SEQ ID NO: 281 - protein, 9 aa, Mus musculus
IAPKKEQRL

SEQ ID NO: 282 - protein, 9 aa, Mus musculus
IPAHNRQTL

SEQ ID NO: 283 - protein, 9 aa, Mus musculus
VPVEPIPSL

SEQ ID NO: 284 - protein, 9 aa, Mus musculus
AYQKIKQAL

SEQ ID NO: 285 - protein, 9 aa, Mus musculus
QGPPYAESL

SEQ ID NO: 286 - protein, 9 aa, Mus musculus
NSPSLQAHL

SEQ ID NO: 287 - protein, 9 aa, Mus musculus
NGPAFVSQV

SEQ ID NO: 288 - protein, 9 aa, Mus musculus
LYSKLPSKL

SEQ ID NO: 289 - protein, 10 aa, Mus musculus
QGPQRYGNRF

SEQ ID NO: 290 - protein, 9 aa, Mus musculus
SGPSAPPKI

SEQ ID NO: 291 - protein, 15 aa, Mus musculus
TKECRAAIAPKKEQR

SEQ ID NO: 292 - protein, 15 aa, Mus musculus
RQTLQSTELQKSQQE

SEQ ID NO: 293 - protein, 15 aa, Mus musculus
SIQRVPVEPIPSLPP

SEQ ID NO: 294 - protein, 15 aa, Mus musculus
YQKIKQALLTAPALR

SEQ ID NO: 295 - protein, 15 aa, Mus musculus
GPPYAESLPCVVRQP

SEQ ID NO: 296 - protein, 15 aa, Mus musculus
SPSLQAHLQALQAVQ

SEQ ID NO: 297 - protein, 15 aa, Mus musculus
GPAFVSQVSQSVAKL

SEQ ID NO: 298 - protein, 15 aa, Mus musculus
LPSKLIQSTSSTSPR

SEQ ID NO: 299 - protein, 15 aa, Mus musculus
NRFVRTQEAAREATQ

SEQ ID NO: 300 - protein, 15 aa, Mus musculus
SSKLTVAVAQSDAAG

(19) United States
(12) Patent Application Publication
(10) Pub. No.: US 2025/0054576 A1
(43) Pub. Date: Feb. 13, 2025

(54) Personalized cancer therapy targeting normally non-expressed sequences

(71) Applicant: Evaxion Biotech A/S, Hørsholm (DK)

(72) Inventors: Christian Garde, Hørsholm (DK); Thomas Trolle, Hørsholm (DK); Jens Vindahl Kringelum, Hørsholm (DK); Pablo Garces, Hørsholm (DK); Emma Christine Jappe, Hørsholm (DK)

(21) Appl. No.: 18/719,468

(22) PCT Filed: Dec. 16, 2022
(86) PCT No.: PCT/EP2022/086444

§ 371 Date: Jun. 13, 2024

(30) Foreign Application Priority Data
Dec. 17, 2021 (EP) 21215594.9
Jul. 28, 2022 (EP) 22187561.0

(51) Int. Cl. G16B 20/30; C12Q 1/6886; G16B 15/30; G16H 20/17

(52) U.S. Cl. CPC G16B 20/30; C12Q 1/6886; G16B 15/30; G16H 20/17

(57) ABSTRACT

Systems and methods for use in cancer immunotherapy. The systems and methods may include designing and producing personalized anti-cancer vaccines which target expression products of genomic sequences, which are not or to a limited degree expressed in normal tissues, but which are found in individual patients' cancer tissue. The systems and methods may include identifying immunogenic amino acid sequences in a sample of malignant tissue from a patient. The systems and methods may include a method for stratifying cancer patients into groups of those eligible for immunotherapy or not, based on their burden of expression of endogenous retrovirus.

### FIELD OF THE INVENTION

The present invention relates to the field of cancer immunotherapy. In particular, the present invention relates to improved means and methods for designing and producing personalized anti-cancer vaccines which target expression products of genomic sequences, which are not or only to a very limited degree expressed in normal tissues, but which are found in individual patients' cancer tissue. Also, the invention relates to a method for treatment of cancer as well as a computer system. In addition, the invention relates to a method for stratifying cancer patients.

### BACKGROUND OF THE INVENTION

Treatment of malignant neoplasms in patients has traditionally focussed on eradication/removal of the malignant tissue via surgery, radiotherapy, and/or chemotherapy using cytotoxic drugs in dosage regimens that aim at preferential killing of malignant cells over killing of non-malignant cells.

In addition to the use of cytotoxic drugs, more recent approaches have focussed on targeting of specific biologic markers in the cancer cells in order to reduce systemic adverse effects exerted by classical chemotherapy. Monoclonal antibody therapy targeting cancer associated antigens has proven quite effective in prolonging life expectance in a number of malignancies. While being successful drugs, monoclonal antibodies that target cancer associated antigens or antigen can by their nature only be developed to target expression products that are known and appear in a plurality of patients, meaning that the vast majority of cancer specific antigens cannot be addressed by this type of therapy, because a large number of cancer specific antigens only appear in tumours from one single patient, cf. below.

As early as in the late 1950'ies the theory of immunosurveillance was formulated and suggested that lymphocytes recognize and eliminate autologous cells—including cancer cells—that exhibit altered antigenic determinants, and it is today generally accepted that the immune system inhibits carcinogenesis to a high degree. Nevertheless, immunosurveillance is not 100% effective and it is a continuing task to develop cancer therapies where the immune system's ability to eradicate cancer cells is sought improved/stimulated.

One approach has been to induce immunity against cancer-associated antigens, but even though this approach has potential, it suffers the same drawback as antibody therapy that only a limited number of antigens can be addressed.

Many, if not all, tumours express mutations. These mutations potentially create new targetable antigens (neoantigens), which are potentially useful in specific T cell immunotherapy if it is possible to identify the neoantigens and their antigenic determinants (neoepitopes) within a clinically relevant time frame. Since it with current technology is possible to fully sequence the genome of cells and to analyse for existence of altered or new expression products, it is possible to design personalized vaccines based on neoantigens and their neoepitopes.

Multiple bioinformatic pipelines hence exist for predicting/identifying neoepitopes from patient derived sequencing data (cf. Hundal, J. et al. 2016; Bjerregaard, A. M. et al. 2017; Bais, P. et al. 2017; Rubinsteyn, A. et al. 2017; Schenck, R. O. et al. 2019). Each pipeline takes a different set of features into account when selecting or ranking neoepitopes, underscoring that the neoepitope selection problem is still unsolved.

International patent application PCT/EP2021/071380 discloses methods for selection of epitopes to include in individualized cancer vaccines; focus is put on identification and utilisation neoepitopes encoded by somatic variants of expressed genes in cancer cells. The methods disclosed in PCT/EP2021/071380 hence rely on an identification of short peptides present in expression products that differ from the normal expression products in the patient, and as such the method in PCT/EP2021/071380 will always require an individual evaluation of the potential usefulness of such short peptides.

### Endogenous Retroelements and Endogenous Viral Elements

Endogenous retroelements (EREs) represent a substantial proportion of the host genome, constituting up to 43% and 37% of the human and mouse genomes, respectively. The vast majority (70-80% of all endogenous retroelements) comprises elements that lack long-terminal repeats (LTRs). In this group we find short and long interspersed retrotransposable elements (SINE and LINE) and are collectively known as non-LTR elements. The remaining endogenous retroelements comprise LTR-bound elements comprising two major groups occupying comparable fractions of the genome: endogenous retroviruses (ERVs) and mammalian apparent LTR retrotransposons (MaLRs) (Kassiotis & Stoye, 2016). From the large group of endogenous retroelements, there is a subset of genes referred to as endogenous viral elements (EVEs), which is a result of an in silico filtering process based on the presence of viral motifs in the gene. As such, this group is mostly composed of ERVs, but can also contain members of the other different subcategories.

The retroviral life cycle is characterized by reverse transcription of the retroviral RNA genome followed by cDNA integration into the host nuclear DNA, where they can persist in the form of a stable integrated provirus. Retroviral infections of early embryonic and germ-line cells can be inherited by subsequent generations and such ancient proviral relics found in the genome comprise what we today known as ERVs. Since the discovery of the first human endogenous retrovirus (hERV) in 1981, more than 400,000 hERV fragments have been found in the human genome, contributing approximately to 5% of human DNA.

ERVs are recognized by their similarity in genomic structure with all retroviruses, typically consisting of a Gag, Pro, Pol and Env genes flanked by long-terminal repeats (LTRs), whereas further accessory ORFs are present in more complex endogenous retroviruses. The Gag ORF encodes the various structural components of the virion capsid, whereas the Pro and Pol ORFs encode enzymatic activities that are involved in protein and nucleic acid processing, respectively. Entry of endogenous retrovirus virions into the target cell is achieved by binding of the endogenous retrovirus Env glycoprotein, which is encoded by the Env ORF, to its cellular receptor. Endogenous retrovirus virions encapsidate two copies of viral single-stranded RNA (SSRNA), and the encapsidated RT enzyme carries out their reverse transcription, usually in the cytosol of the target cell (Kassiotis & Stoye, 2016). The newly synthesized cDNA, which is still part of the pre-integration complex, is then transported to the nucleus for integration into host DNA. Thus, endogenous retroviruses are the only endogenous retroelements with the potential to achieve cell to cell infection. However, integrated ERV sequences degenerate over time by accumulation of mutations or recombination events, which destroy their ability to produce infectious virus, yet they may still propagate efficiently in the germ-line through retrotransposition rather than reinfection (Magiorkinis et al., 2012).

The most frequently used classification of hERVs in literature is based on the binding site for the tRNA primer. For example, a retrovirus using lysine (K) tRNA molecules to prime reverse transcription would generate an HERVK gene after genomic insertion. Although this classification was sufficient during the early years of hERV research, the system became obsolete after the discovery of many other hERV families in the human genome.

Based on their similarity to exogenous retroviruses, hERV families are now grouped into three classes. hERVs with homology to mammalian type C retroviruses (gamma retroviruses), such as murine leukemia virus (MLV), have been placed in class I. Class I represents a highly heterogeneous group of hERVs with many different families, and many Class I elements are chimeras composed of segments derived from unrelated retroviruses. Class II consists of hERVs related to mammalian type B and D retroviruses (beta retroviruses) represented by mouse mammary tumour virus (MMTV) and contains most of the genes previously belonging to the HERVK group. This group is also characterized by encompassing all the most recent retroviruses found in the human genome (HERVK10), many of which are human-specific. Class III are characterized by its similarity to human foamy viruses (Spumaviridae) and besides a couple of exceptions, all its members lack env-like ORFs. This group also contains a large number of nonautonomous elements known as mammalian apparent LTR retrotransposons (MaLRs) and THE1. No endogenous counterparts of exogenous lentiviruses such as HIV are known in the human genome (Pavlicek & Jurka, 2006).

### ERV Expression

Even though most, if not all, ERVs have accumulated replication-inactivating mutations, many still contain intact ORFs with the potential of producing retroviral proteins. These ERV-derived proteins may serve as a source of antigen for the immune system (e.g. includes MMTV in mice and HERV-K18 in humans) and for certain ERVs, they even possess a biological function within the host. This evidence suggests that immunological tolerance to ERV-derived proteins is not complete (Kassiotis, 2014).

Some cases have been reported where endogenous retroviruses expression benefit the host. For example, the Fv1 (Friend virus susceptibility 1) and Fv4 loci in mice encode retroviral restriction factors of endogenous retrovirus origin which defend the host from further infection with exogenous retroviruses. Also, the well described members of the syncytin family, which are encoded by genes derived from Env ORFs of endogenous retroviruses, are essential for placental development, possibly modulating fetomaternal tolerance. This evidence highlights the possible interdependence of ERV derived genes of a host species and its reproductive success (Dupressoir et al., 2012).

ERV activity in a cell is regulated both by cell-intrinsic factors and external signals and is therefore neither ubiquitous nor constitutive. Epigenetic silencing seems to be a major mechanism in which the cell prevents transcription of repetitive elements (including ERVs), especially in the germ plasm and the early embryo (Kassiotis, 2014). Nevertheless, a sizeable proportion of endogenous retroelements may still be transcribed in adult cells and tissues. This expression follows a tissue-specific pattern, often as a result of co regulation with tissue-defining host genes (Young et al., 2014).

Somatic expression of endogenous retroelements is also regulated by various environmental stimuli that affect either DNA and histone methylation in earlier develop-mental windows or the balance of transcription factor networks in a given somatic cell type. External cues may involve the xenobiotic response element (XRE) pathway activation, immune cell activation and dietary restrictions (Kassiotis & Stoye, 2016).

In humans, ERVs from different families have been reported to be (over) expressed in both tumour samples as well as in cancer cell lines. In a cohort of 89 patients with both ovarian cancer and benign ovarian diseases, cancer patients showed increased mRNA levels of HERV-K, HERV-R and HERV-E when compared to healthy controls, and its expression can be associated to poor prognosis (Iramaneerat et al., 2011; Wang-Johanning et al., 2007). Other cancers have also been related to HERV-K (over)expression, like renal carcinoma, lymphoma, leukaemia, melanoma, sarcoma, endometrial or lung cancer. Moreover, even if HERV-K is the most extensively researched human ERV group, other ERVs may also have a role in oncogenesis, especially HERV-H and HERV-W (Vergara Bermejo et al., 2020).

### ERV Immunomodulation

Several endogenous retrovirus-derived Env ORFs with full coding capacity seem to have been retained in the murine and human genomes, suggesting that they have further physiological roles that prevent the disintegration of these endogenous retroelements, even over extended time periods. Moreover, host genomes harbour recently integrated groups of endogenous retroviruses that evolution has not yet irreversibly damaged, although replication of such proviruses in humans is not known to occur. Finally, epigenetic control of endogenous retroviruses is primarily important in protecting germline cells from excessive reinfection or transposition, whereas somatic cells are susceptible to endogenous retrovirus reactivation. Together, these properties of endogenous retroviruses and the necessity for the host immune system to continue to defend against exogenous infection with retroviruses, as well as other pathogens, prevent complete innate and adaptive immunological tolerance of endogenous retroviruses. In fact, a large body of evidence suggests that immune responses to endogenous retroviruses can be induced, often with pathological consequences, particularly related to autoimmune diseases. Different mechanisms have been described which link hERVs to some chronic diseases, such as an undesired immune reaction against self-nucleic acids based on reverse-transcribed cDNA intermediates of endogenous retroelements (Balada et al., 2009; Volkman & Stetson, 2014).

Endogenous retroelements can play a dual role in immunity tuning, as they can influence immune reactivity against themselves but can also play a role in immunosuppression. The latter becomes evident based on the advantageous features that enhanced ERV expression provides during cancer development. Essays with interference RNA showed that silencing of and Env gene in B16 murine melanoma resulted in tumour rejection under conditions where control melanoma cells grow into lethal tumours, evidencing its role in tumour T-cell mediated immune escape in vivo (Mangeney et al., 2005). Similar evidence was observed on human breast cancer, pancreatic cancer and prostate cancer, where Env protein also proved to be essential for tumourigenesis and metastasis of cancer cells lines. Knockdown of HERV-K Env impaired the expression of tumour-associated genes including Ras, p-RSK, p-ERK and p-AKT resulting in smaller tumours (Li et al., 2017; Zhou et al., 2016). In accordance with what is now established for HIV, research has determined that the immunosuppressive effect of Env proteins is linked to the transmembrane domain, which is called p15E for the Murine Leukemia Virus (MuLV) gene in mice (Scheeren et al., 1992). This domain is also known as immunosuppressive domain (ISD) and is shown to inhibit activation of PBMCs and modulate cytokines release as well as gene expression. This immune regulation has not only been evidenced for HERV-K, but for HERV-W, HERV-H and HERV-FDR as well, where its expression has been detected in placenta and implicated in tolerance against the semi-allogenic fetus though dendritic cell mediated suppression of T-cell activation (Hummel et al., 2015; Lokossou et al., 2020; Morozov et al., 2013).

Alternatively, induction of an IFN response or cellular activation by replication intermediates of endogenous retroelement nucleic acids inevitably heightens immune reactivity to unrelated antigens. By mimicking viral infection, endogenous retroelements may provide the necessary ‘intrinsic adjuvant’ for the immune response against poorly immunogenic targets. For example, induced ERV expression following treatment with azacytidine, an inhibitor of DNA methylation, induces an IFN response which in turn increased tumour cell immunogenicity

(Chiappinelli et al., 2015; Roulois et al., 2015). This induction of viral mimicry by ERVs gets an additional level of complexity when factoring the presence of multiple copies of endogenous retroelements of any given group. The obligatory hijacking or ‘accidental’ sharing of protein products between distinct groups of endogenous retroelements, underscores the marked potential of distinct endogenous retroelement loci to interact with one another.

Consequently, the potential of individual endogenous retroelements to induce an immune response depends on their expression but can be further influenced by the combination of interacting retroelements that are expressed in a given cell type.

### ERVs Role on Cancer

The fact that infectious retroviruses can cause cancer through insertional mutagenesis initially reinforced the concept that ERVs are causative agents of many cancers. However, this notion has not stood up to experimental scrutiny. The recent greater appreciation of the complexity of ERV biology and the identification of dedicated host mechanisms controlling ERV activity have revealed novel interactions between ERVs and their hosts with the potential to cause or contribute to disease.

There are numerous examples of ERV protein expression or even virion production in mouse and human cancer cells. However, both ERV proteins and virions can be seen also in either certain healthy tissues, such as the placenta, or in infection and other non-neoplastic diseases. Therefore, elevated ERV activity, even if restricted to cancer cells, should not be taken to signify causality. Rather, most of this activity is likely to represent lack of ERV regulation under these conditions (Kassiotis, 2014).

Global DNA hypomethylation, leading to epigenetic de-repression is a hallmark of cellular transformation and a major contributor to oncogene activation. In the altered epigenetic landscape of transformed cells, ERV de-repression should be expected as a consequence (Szpakowski et al., 2009). Characteristic patterns of ERV expression are often seen in various tumours, offering a unique approach to immunotherapy. Tumour-restricted expression has been proposed for several ERVs, including HERV-K (HML6) in melanomas, HERV-K (HML2) in germ-cell carcinomas, HERV-E in renal cell carcinomas, or even Syncytin-1 in diverse cancers. HERV expression in some of these cases has also been demonstrated to lead to immune reactivity against hERV-derived epitopes (Kassiotis, 2014). For example, a recent study from Saini et al. constructed a library of 1169 peptides originating from 66 previously identified hERV loci that potentially retained translational activity, and observed enriched hERV-directed T cells in patients with myeloid malignancies when compared to healthy donors (Saini et al., 2020).

Cancer-specific transcription of ERVs not only accompany activation of oncogenic pathways, but it also leads to the generation of exosomes resembling VLPs that can express Gag and Env. The presence of the ERV proteins in these particles will elicit innate and adaptive immune responses, being able to generate specific CD8+ T-cells among other immune responses. Similarly, the viral proteins processed by the cancer cells would be presented by the MHC-I or -II depending on them being processed through the proteasome or in the endosome. However, these cancer-induced immune responses are not able to control tumour development, probably influenced to some extent by the immunosuppressive state generated by the ISD in the p15E Env protein. A further activation of the immune system appears to be needed to fight the tumour (Vergara Bermejo et al., 2020).

### ERVs Immunogenicity

ERVs have been detected in several cancers, while they remain largely silent in healthy tissues. Their low immunogenicity together with their immunosuppressive capacity aid cancer to escape immunosurveillance. It has been a subject of debate, which, if any, of the specific

ERV types can be found with sufficient cancer specificity to be targeted (Vergara Bermejo et al., 2020). HERVs have been tested as cancer targets for several decades, applying a variation of delivery methods (dendritic cell pulsing and CAR-T cells targeting a specific hERV) (Vergara Bermejo et al., 2020). Typically, these studies only target a single or very few hERVs simultaneously; hERVs that have demonstrated cancer-specificity in the sense that have been identified as common between patients grouped based on a single cancer type.

ERVs comprise about 4.7% of the mouse genome, and unlike what is known in humans, the murine genome still contains replication competent ERVs which can produce functional infectious virions: the MuLV and MMTV genes. By the beginning of the 2000s, expression of these genes had already been reported in several murine cell lines as well as their presence as MHC class I restricted T-cell antigens, but no proof of its potential for tumour treatment in in vivo models.

One of the earliest pieces of evidence of effective tumour immunization using ERVs was provided by the Takeda group, where a DNA vaccine encoding a B-gal/gp70 fusion protein (but not gp70 alone) could induce protective immunity against CT26 challenge (Takeda et al., 2000). Matching results were also obtained by Jason Rice et al., who reported that the administration of a DNA vaccine encoding gp70 alone was a poor inducer of CTL, but its effect was significantly improved leading to rapid CTL induction when administering a fusion protein containing minimal gp70 sequence (AH1 peptide, sequence SPSYVYHQF) together with minimized domain of a pathogen-derived sequence from tetanus toxin (fragment C (FrC) minimal domain (DOM)) (Rice et al., 2002).

An additional report using a mouse ERV (mERV) DNA vaccination against H-2b and H-2d haplotype mice only presented a strong immune response when administered with a CD40 agonist. However, the timing when this agonist was administered appeared to be of great relevance and proved to be most effective during tumour rejection, as it could even be deleterious during the priming of the immune response (Bronte et al., 2003). There are few reports using ERVs purified proteins as a treatment strategy, but the study performed by Caseres et al., albeit with a different approach, agrees with the formerly presented results. In this study, they demonstrated that an IFA emulsion containing a combination of purified AH1 peptide with a Th1 stimulant peptide (OVA (323-337)) protected 83% of BALB/c mice from a CT26 challenge, unlike the emulsion containing only AH1 which had no protective effect (Casares et al., 2001).

Another strategy that has been employed for ERV-mediated immunization is chimeric antigen receptor (CAR) T-cell therapy. The study performed by Krishnamurthy et al. associated HERV-K gene to a TAA-like target after having identified the expression of HERV-K protein in 220 melanoma samples (with various stages of disease) and not on 139 normal organ donor tissues using immunohistochemical (IHC) analysis. Observations on in vivo adoptive transfer of CAR+ T cells generated using the sleeping beauty (SB) system (a non-viral approach that utilizes a transposon and transposase) resulted in lysed tumour cells in vitro and anti-tumour effect in vivo in an antigen-specific manner (Krishnamurthy et al., 2015).

One of the most popular strategies used for ERV immunization in public literature to date has been through virus like particles (VLPs) platform, likely due to their size, repetitive surface pattern, capacity to generate both innate and adaptive immune response, as well as being a safe and economically profitable system (Qian et al., 2020). Kershaw et al. presented the first evidence on the efficacy of recombinant vaccinia immunization encoding gp70 minimal determinant (AH1 peptide), which significantly protected mice from subsequent CT26 tumour challenge but proved ineffective against stablished tumours (Kershaw et al., 2001). The latter could only be achieved when either gp70 or p15E peptide was pulsed onto in vitro-generated bone marrow-derived DCs, which were subsequently used to treat mice. Similar results have been shown for lung cancer, where the injection of a modified vaccinia Ankara (MVA) encoding HERV-K Env and Gag prevented lung tumour outgrowth and metastasis in mice (Kraus et al., 2013, 2014).

Over the last couple of years, additional body of evidence have further demonstrated ERV-based therapy efficacy by means of VLPs. A vaccination strategy designed to induce both humoral as well as cellular immune response against a MuLV virus-like particles was performed through the administration of an adenovirus type 5 encoding the melanoma-associated retrovirus (MelARV) proteins Gag and Env. Despite a lack of antibody induction, they found that T cell responses were strong enough to prevent colorectal CT26 tumour growth and progression in 57% of BALB/c mice after a single vaccination, both before and after tumour challenge. The protective efficacy further increased when combined with checkpoint inhibitor therapy, leading to complete tumour regression (Neukirch et al., 2019). This protection appeared to be long-term, as none of the mice that survived the initial CT26 tumour challenge developed tumours after a rechallenge with 4T1 tumour cells.

Peltonen et al. also obtained positive results using ERVs as therapeutic targets through a similar delivery platform called Peptide-coated Conditionally Replicating Adenovirus (PeptiCRAd) but using different mERV targets and on an aggressive triple negative breast cancer model (4T1). The vaccine platform was complexed with immunopeptidomics-identified mERV targets FYLPTIRAV and TYVAGDTQV peptides (Q811J2 Uniprot accession). These peptides can be mapped to a multitude of putative mERV ORFs but not to the well described MuLV gene. The treatment with ERVs showed statistically significant protection when compared to the virus alone, but not as evident as the results shown above. In this approach, combination therapy with PD-1 did not increase the level of protection (Peltonen et al., 2021).

WO 2021/005339 discloses cancer-specific LTR element-spanning RNA transcripts, which are associated with small cell lung cancer and/or melanoma, and also discloses use of expression products from these transcripts in active specific cancer immunotherapy.

### Object of the Invention

It is an object of embodiments of the invention to provide means and methods for the identification and utilisation of nucleic acid sequences and their expression products, where the nucleic acid sequences in normal cells are not expressed or expressed at very low frequencies in random normal tissue samples.

### SUMMARY OF THE INVENTION

Based on baseline expression (RNAseq) data of >6,000 human healthy tissue samples from TCGA, the present inventors have identified a substantial number of EVE sequences that are non-expressed or expressed at very low levels across all normal tissues and which at the same time are potentially ideal targets in a personalized cancer immunotherapy. As the majority of the identified EVEs can be defined as ERVs, ERVs will be generally used as the term to denote these sequences below even though a minor fraction might not strictly speaking be ERVs.

When >400 malignant melanoma biopsies were studied, several patient-specific ERV sequences that are overexpressed were identified.

Applying a set of HLA ligand prediction tools, HLA-binding ligands that have high ligand probability in binding to patient-specific HLA molecules were identified from the full-length ERV sequences, and it was demonstrated that expression of these ERV sequences in non-malignant tissues was at most marginal.

Thus, the invention generally relates to a method that involves a bioinformatics approach to identify an extensive baseline expression profile from which patient-specific ERVs and HLA-specific ligands comprised therein can be identified for each individual patient and administered to the patient as a personalized therapy.

An attractive feature of this approach is that the ERV sequences are known in advance, meaning that a patient's cancer transcriptome merely has to be queried for the presence of RNA transcripts of the selected ERV sequences, which have in advance been screened for off-target activity.

The present inventors have applied a personalized approach to ERVs, thus predicting ligands from patient-specific (overexpressed) ERVs for each patient in the clinic, rather than applying a traditional “warehouse” approach as in WO 2021/005339, where the same ERVs are used across multiple patients based on previous experience, where their expression has been correlated with specific cancer types.

So, the invention relates in a 1^{st} aspect to a method for identifying immunogenic amino acid sequences in a sample of malignant tissue from a patient (preferably human) comprising
A) determining amino acid sequences of proteinaceous expression products from the malignant tissue,
B) analysing said amino acid sequences to identify therein proteinaceous expression products of selected genomic sequences in the patient's species,
C) identifying-in the proteinaceous expression products-the amino acid sequences, which are those that will bind to MHC (in humans: HLA) molecules of the patient, where said selected genomic sequences constitute a subset of all sequences of the genome of said species and where said subset is constituted by sequences, which encode proteinaceous expression products in at most 5% of samples from any healthy tissue, where said healthy tissue is a tissue of a type found in the patient, and where said healthy tissue optionally does not include testis tissue and/or brain tissue.

In a 2^{nd} aspect, the invention relates to a method of treating a malignant neoplasm in a patient, preferably a human patient, the method comprising sequences in a sample of malignant tissue from a patient (preferably human) comprising
A) determining amino acid sequences of proteinaceous expression products from the malignant tissue,
B) analysing said amino acid sequences to identify therein proteinaceous expression products of selected genomic sequences in the patient's species,
C) identifying—in the proteinaceous expression products—the amino acid sequences, which are those that will bind to MHC (in humans: HLA) molecules of the patient, where said selected genomic sequences constitute a subset of all sequences of the genome of said species and where said subset is constituted by sequences, which encode proteinaceous expression products in at most 5% of samples from any healthy tissue, where said healthy tissue is a tissue of a type found in the patient, and where said healthy tissue optionally does not include testis tissue and/or brain tissue, and
D) administering to the patient one or more peptides identified in step C or one or more polypeptides comprising 2 or more peptides identified in step C or one or more expression vectors encoding and capable of expressing said one or more peptides identified in step C or expressing one or more polypeptides comprising 2 or more peptides identified in step C so as to induce a specific adaptive immune response against said one or more peptides, where said selected genomic sequences constitute a subset of all sequences of the genome of the patient's species and where said subset is constituted by sequences, which encode proteinaceous expression products in at most 5% of samples from healthy tissue, where said healthy tissue is a tissue of a type found in the patient, and where said healthy tissue optionally does not include testis tissue and/or brain tissue.

In a closely related aspect, the invention relates to the peptides identified in step C for use in a therapeutic method, in particular in the therapeutic method of the 2^{nd} aspect of the invention.

In a 3^{rd} aspect, the invention relates to a computer or computer system for identifying immunogenic amino acid sequences in a sample of malignant tissue from a patient, said computer or computer system comprising
1) an input component for inputting amino acid sequence or mRNA data;
2) optionally executable code for determining amino acid sequences from mRNA data;
3) a database comprising amino acid sequences of expression products of genomic sequences;
4) executable code for identifying presence—in inputted amino sequences or amino acid sequences encoded by inputted mRNA—of sequences present in the amino acid sequences in the database;
5) executable code that identifies and optionally ranks amino acid sequences identified by the executable code in 4 in accordance with their predicted ability to bind a selection of MHC molecules; and 6) a component for outputting or storing the amino acid sequences identified and/or ranked
wherein said genomic sequences in 3 constitute a subset of all sequences of the genome of a species and where said subset is constituted by sequences, which encode proteinaceous expression products in at most 5% of samples from any healthy tissue type found in the species, where said healthy tissue type optionally does not include testis tissue and/or brain tissue.

In a 4^{th} aspect, the invention relates to a method for determining whether a cancer patient is likely to benefit from anti-cancer immunotherapy, comprising determining the number of EVEs, such as ERVs, which are expressed in said patient, and categorizing the cancer patient as being likely to benefit from the anti-cancer immunotherapy if said number of expressed EVEs or ERVs exceeds a predefined threshold.

### LEGENDS TO THE FIGURES

FIG. 1 shows a graph depicting the response rates in patients treated with high and low tumour mutational burden (TMB), respectively.

FIG. 2 shows a greytone heatmap of the expression of 9 tumour specific antigens (TSAs) across various tissue samples. Black indicates expression in 0% of tissue samples from the tissue in question, white indicates 50% of tissue samples, cf. the greytone designation at the right.

FIG. 3 shows a greytone heat map of the expression of 12,202 selected (i.e. included) hERV expression products across various tissue samples. Black indicates expression in 0% of tissue samples from the tissue in question, white indicates 50% of tissue samples, cf. the greytone designation at the right.

FIG. 4 shows a greytone heat map of the expression of 21,764 deselected (i.e. excluded from the set of potential target sequences) hERV expression products across various tissue samples. Black indicates expression in 0% of tissue samples from the tissue in question, white indicates 50% of tissue samples, cf. the greytone designation at the right.

FIG. 5 is a bar graph showing expression in fractions of healthy tissue samples of TSAs (not including brain and testis), selected/included ERVs, and deselected/excluded ERVs (corresponding to FIG. 3), respectively.

FIG. 6 is a bar graph showing expression in fractions of healthy tissue samples of TSAs (not including brain and testis), selected/included ERVs, and deselected/excluded ERVs (corresponding to FIG. 3, but allowing expression in brain and testis), respectively.

FIG. 7 shows bar graphs depicting the numbers of patients having high ERV burden (>50 ERVs) and low ERV burden (<50 ERVs) in groups of patients having high TMB and low TMB, respectively, derived from 3 published scientific studies.

FIG. 8 shows line graphs of observed patient survival over time in patient groups having high or low ERV burden from 3 different published scientific studies.

FIG. 9 shows line graphs of observed patient survival over time in high TMB patient groups having high or low ERV burden from 3 different published scientific studies.

FIG. 10 shows line graphs of observed patient survival over time in low TMB patient groups having high or low ERV burden from 3 different published scientific studies.

FIG. 11 shows line graphs of observed patient survival over time for patients with high and low ERV burden in patient groups with high or low TMB, respectively.

FIG. 12 shows graphs relating immunization against ERV encoded expression products and the in vivo protective effect against tumours.
A: Schematic depiction of immunization scheme.
B: Tumour growth in 3 groups of mice vaccinated expressed as volume vs. time.
C: Tumour growth in 3 groups of mice vaccinated expressed as volume vs. time.
D: Tumour growth in 3 groups of mice vaccinated expressed as area under curve (AUC).

To test whether ERVs can be relevant targets for immunotherapy, groups of mice were vaccinated intramuscularly (i.m.) with PR-ERVs, MS-ERVs or mock pDNA in one-week intervals and in a vaccine administration scheme comprising two EP-based prime immunizations followed by three poloxamer-based ones (FIG. 12A). Immunizations commenced two weeks prior to subcutaneous (s.c.) challenge with a tumourigenic dose of CT26 cancer cells. In contrast to mock pDNA-treated mice that developed tumours of significant end volume (FIG. 12B), mice vaccinated with PR-ERVs and MS-ERVs demonstrated strong prevention of CT26 tumour establishment (FIGS. 12B and 12C).

FIG. 13 shows two graphs relating threshold of hERV numbers relative to Hazard ratios. Top graph shows the relation for the full hERV database, and the bottom graphs shows the relation for the list of hERVs provided in the table in Example 4.

FIG. 14. shows a bar graph, providing the numbers of samples from 13 different cancers.
SKCM: Skin Cutaneous Melanoma
LUAD: Lung adenocarcinoma
LUSC: Lung squamous cell carcinoma
BLCA: Bladder Urothelial Carcinoma
COAD: Colon adenocarcinoma
STAD: Stomach adenocarcinoma
THCA: Thyroid carcinoma
BRCA: Breast invasive carcinoma
GBM: Glioblastoma multiforme
KIRC: Kidney renal clear cell carcinoma
LIHC: Liver hepatocellular carcinoma
KIRP: Kidney renal papillary cell carcinoma
ESCA: Esophageal carcinoma

FIG. 15 shows a graph relating TMB to EVE burden for the cancers set forth in FIG. 14.

### DETAILED DISCLOSURE OF THE INVENTION

### Definitions

An endogenous retroelement (ERE) is a genetic element. EREs constitute nearly 50% of the human genome. These elements are present in almost all organisms and believed to be remnants of transposable elements that integrated in germline cells millions of years ago. Most ERE sequences contain mutated or truncated open reading frames and have lost their capacity to transpose in the genome. EREs comprise short and long interspersed retrotransposable elements (SINE and LINE), and these are collectively known as non-LTR elements. The remaining endogenous retroelements comprise LTR-bound elements comprising two major groups occupying comparable fractions of the genome: endogenous retroviruses (ERVs) and mammalian apparent LTR retrotransposons (MaLRs) (Kassiotis & Stoye, 2016).

An “endogenous viral element” (“EVE”) is an ERE, which is member of a subset of genes, which is a result of an in silico filtering process based on the presence of viral motifs in the gene. As such, this group is mostly composed of ERVs, but can also contain members of the other different subcategories.

A “novel or unannotated open reading frame” (abbreviated a “nuORF”) is a genomic sequence, which is not conventionally the source of a translated product, but where immunopeptidomic analyses have revealed the existence of MHC binding peptides derived from malignant tissue (Ouspenskaia T et al. 2021, Nature Biotechnology, doi.org/10.1038/s41587-021-01021-3).

A “malignant neoplasm” (also termed a cancer or malignant tumour) denotes a group of cells in a multicellular organism, which exhibit uncontrolled growth, invasive growth, and, normally, the ability to metastasize.

A “cancer specific” antigen, is an antigen, which does not appear as an expression product in an individual's non-malignant somatic cells, but which appears as an expression product in cancer cells in the individual. This is in contrast to “cancer-associated” antigens, which also appear—albeit at low abundance—in normal somatic cells, but are found in higher levels in at least some malignant tumour cells. In general, the peptides identified according to the present invention are considered to be cancer specific.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

An MHC molecule (major histocompatibility molecule) is a tissue antigen expressed by nucleated cells in vertebrates, which binds to peptide antigens and displays (“presents”) the antigens to T-cells carrying T-cell receptors. MHC class I is expressed by all nucleated cells and primarily present proteolytically degraded protein fragments derived from proteins present in the cell. MHC class II is expressed by professional antigen presenting cells that typically take up extracellular protein, degrade it with lysosomal proteases, and present protein fragments on the surface. In humans, the MHC molecules are known as human leukocyte antigens (HLA), which in the present invention are the preferred MHC molecules to evaluate binding to.

A “T-cell epitope” is an MHC binding peptide, which is recognized as foreign (non-self) by a T-cell in a vertebrate due to specific binding between a T-cell receptor and the cell carrying the MHC-peptide complex on its surface. Hence, a peptide, which constitutes a T-cell epitope in one individual will not necessarily be a T-cell epitope in a different individual of the same species. First of all, two individuals having differing MHC molecules that bind different sets of peptides, do not necessarily present the same peptides complexed to MHC, and further, if a peptide is autologous in one of the individuals it may not be able to bind any T-cell receptor.

A “neoepitope” is an antigenic determinant (typically an MHC Class I or II restricted epitope), which does not exist as an expression product from normal somatic cells in an individual due to the lack of a gene encoding the neoepitope, but which exists as an expression product in mutated cells (such as cancer cells) in the same individual. As a consequence, a neoepitope is from an immunological viewpoint truly non-self in spite of its autologous origin and it can therefore be characterized as a tumour specific antigen in the individual, where it constitutes an expression product. Being non-self, a neoepitope has the potential of being able to elicit a specific adaptive immune response in the individual, where the elicited immune response is specific for antigens and cells that harbour the neoepitope. Neoepitopes are on the other hand specific for an individual as the chances that the same neoepitope will be an expression product in other individuals is minimal. Several features thus contrast a neoepitope from e.g. epitopes of tumour specific antigens: the latter will typically be found in a plurality of cancers of the same type (as they can be expression products from activated oncogenes) and/or they will be present—albeit in minor amounts—in non-malignant cells because of over-expression of the relevant gene(s) in cancer cells.

A “neopeptide” is a peptide (i.e. a polyamino acid of up to about 50 amino acid residues), which includes within its sequence a neoepitope as defined herein. A neopeptide is typically “native”, i.e. the entire amino acid sequence of the neopeptide constitutes a fragment of an expression product that can be isolated from the individual, but a neopeptide can also be “artificial”, meaning that it is constituted by the sequence of a neoepitope and 1 or 2 appended amino acid sequences of which at least one is not naturally associated with the neoepitope. In the latter case the appended amino acid sequences may simply act as carriers of the neoepitope, or may even improve the immunogenicity of the neoepitope (e.g. by facilitating processing of the neopeptide by antigen-presenting cells, improving biologic half-life of the neopeptide, or modifying solubility).

The term “amino acid sequence” is the order in which amino acid residues, connected by peptide bonds, lie in the chain in peptides and proteins. Sequences are conventionally listed in the N to C terminal direction.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right-typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens. In the present context the immune responses induced by the peptides identified are

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “polypeptide” is in the present context intended to mean both short peptides of from 2 to 50 amino acid residues, oligopeptides of from 50 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups

Embodiments of the 1st aspect of the invention

The first aspect relates to a method for identifying immunogenic amino acid sequences in a sample of malignant tissue from a patient comprising
A) determining amino acid sequences of proteinaceous expression products from the malignant tissue,
B) analysing said amino acid sequences to identify therein proteinaceous expression products of selected genomic sequences in the patient's species,
C) identifying—in the proteinaceous expression products—the amino acid sequences, which are those that will bind to MHC molecules of the patient,
where said selected genomic sequences constitute a subset of all sequences of the genome of said species and where said subset is constituted by sequences, which encode proteinaceous expression products in at most 5% of samples from any healthy tissue, where said healthy tissue is a tissue of a type found in the patient, and where said healthy tissue optionally does not include testis tissue and/or brain tissue.

By focussing on expression of genomic sequences in the patient's healthy tissues, it is possible to avoid or minimize adverse effects caused by induction of immunity against healthy tissue. It will be understood that the healthy tissue in question normally will be a reference tissue where samples from multiple sources have been investigated for expression of the genomic sequences. Hence, as a rule, the expression profile in the patient's own normal tissue is not determined, but by ruling out that expression of the genomic sequences take place in >5% of multiple tissue samples from other sources, the risk of inducing adverse events is reduced significantly.

It is be noted that the present approach automatically takes into consideration the age and sex of the patient: for instance, if the patient is female, it is irrelevant if the amino acid sequences identified in the malignant tissue are expressed in >5% of samples from males and vice versa.

In addition, the method allows for inclusion in identified amino acid sequences of sequences from testis and/or brain—both tissues are immune privileged (in case of the brain due to the blood-brain barrier) in the sense that immune responses induced against testis-specific or brain-specific antigens are unlikely to be harmful to the patient. So, in some embodiments, the healthy tissue does not include testis tissue; brain tissue; or testis and brain tissue. On the other hand, in the most safe embodiments, the healthy tissue includes testis and brain tissue—this has the consequence that the number of identified amino acid sequences is lowered compared to a situation where expression in these two tissues is ignored in the selection and identification process.

Preferably, the genomic sequences are selected from endogenous retroelement (EVE) sequences, such as ERV sequences, nuOFR sequences, and genomic sequences that are transcribed as alternatively spliced sequences, but in essence any genomic sequences can be pre-selected if they are considered to be likely to be expressed under certain circumstances and if they contain the necessary MHC binding amino acid stretches. However, the preferred genomic sequences are ERV sequences.

In particularly preferred embodiments, the amino acid sequences of peptides that will bind to MHC molecules of the patient are amino acid sequences that will bind both MHC Class I and MHC Class II molecules of the patient (in the sense that after antigen processing, there will be both binding to MHC Class I and II). This provides amino acid sequences that have a maximum ability to include both humoral and cellular immune responses. However, the amino acid sequences can also be those that bind MHC Class I molecules, but not MHC Class II molecules of the patient, or that bind MHC Class II molecules, but not MHC Class I molecules of the patient.

In order to identify MHC class I binding amino acid sequences, the following algorithms can be used:
netMHCpan-4.1: pubmed.ncbi.nlm.nih.gov/32406916/
MHCflurry: pubmed.ncbi.nlm.nih.gov/32711842/
mixMHCpred: pubmed.ncbi.nlm.nih.gov/28832583/

To identify MHC Class II binding amino acid sequences, the following algorithms can be used
netMHCIIpan-4.0: pubmed.ncbi.nlm.nih.gov/32406916/
mixMHC2pred: pubmed.ncbi.nlm.nih.gov/31611696/
BERTMHC: pubmed.ncbi.nlm.nih.gov/34096999/

It is to be noted the list of selected genomic sequences which ultimately will emerge from the method is influenced by the initial choice of MHC molecules, the binding to which predictions are made for. For instance, since there on a global level are ethnic variations in the frequencies of both HLA Class I and HLA Class II molecules (cf. John M et al. 2010, J. Immunol. 184: 4368-4377 and Pidala J et al. 2012, Bone Marrow Transplant 48 (3): 246-350), the prediction of binding to MHC (HLA) could be optimized relative to the ethnic composition of the population in the relevant area. For instance, the list provided in Example 4 would have a different composition if focus had been on a different set of HLA molecules.

Step A typically comprises determination of the amino acid sequence from mRNA of the patient's malignant tissue, i.e. the mRNA from the malignant tissue is extracted and analysed for the presence of the selected genomic sequences.

As shown in the Examples the selected genomic sequences are identified by determining the expression profile of genomic sequences across a plurality of samples from a plurality of tissues to select those genomic sequences that are expressed in <5% of the plurality of samples. The 5% threshold is arbitrary but is considered a safe threshold, which rules out adverse events in a vast majority of patients. However, in case of e.g. highly malignant cancers, the 5% threshold may be dispensed with, allowing for identification of target sequences, which are expressed in a higher proportion of normal tissue samples from various tissues. On the other hand, if it is desired to minimize the number of potential adverse events, the threshold can be lowered, such as to 4%, 3%, 2% and event to 1% or lower values. The effect is that the number of selected genomic sequences will decrease if the threshold is set lower-so the list of selected genomic hERV sequences provided in Example 4 would be shorter, if a lower threshold would be chosen. Conversely, if the threshold is set higher, the length of the resulting list would be longer.

However, in line with the above, the plurality of samples of a plurality of tissues does in some embodiments not include samples from testis and brain tissue, which provides for a very safe immunization strategy, whereas an even safer approach allows that the plurality of samples of a plurality of tissues includes samples from testis and brain tissue.

### Embodiments of the 2^{nd} Aspect of the Invention

This aspect relates to a method of treating a malignant neoplasm in a patient, preferably a human patient, the method comprising carrying out steps A-C of the first aspect of the invention and any embodiments thereof discussed herein, and subsequently administering to the patient one or more peptides identified in step C or one or more polypeptides comprising 2 or more peptides identified in step C or one or more expression vectors encoding and capable of expressing said one or more peptides identified in step C or capable of expressing one or more polypeptides comprising 2 or more peptides identified in step C so as to induce a specific adaptive immune response against said one or more peptides, where said selected genomic sequences constitute a subset of all sequences of the genome of the patient's species and where said subset is constituted by sequences, which encode proteinaceous expression products in at most 5% of samples from any healthy tissue, where said healthy tissue is a tissue of a type found in the patient, where said healthy tissue optionally does not include testis tissue and/or brain tissue.

In preferred embodiments, the peptides identified in step C, which serve as basis for the administration of peptides/polypeptides/expression vectors, have been identified in a plurality of cancer patients. Such “shared” expression products found in samples from multiple patients can for instance be those found in historical samples from patients, and it is of particular relevance to include such shared expression products that are related to a positive outcome of immune therapy.

The number of peptides, which are identified in step C and form basis for the administration step naturally varies from patient to patient. However, if a polypeptide is administered or an expression vector encoding such a polypeptide is administered, the number of included amino acid sequences of peptides will typically range between 3 and 50. The number will typically be at least 4, 5, 6, 7, 8, 9 or 10 amino acid sequence of peptides identified in step C, and typically at most 45, 40, 35, or 30.

This method is in preferred embodiments provided as part of a combination treatment of the malignant neoplasm, where the patient also receives a treatment selected from the group consisting of other therapeutic cancer vaccination, chemotherapy, radiotherapy, adoptive T-cell therapy (such as CAR-T cell therapy), targeted antibody therapy, cytokine therapy, and immune checkpoint inhibitor therapy. Typically, the other therapeutic cancer vaccination will be vaccination that induces immune responses against neoepitopes or neoantigens but also targeting of cancer-associated antigens can be relevant.

The chemotherapy can be any treatment with cytostatic or cytotoxic compounds, such as treatment with alkylating agents, antimetabolites, anti-microtubule agents, topoisomerase inhibitors, and cytotoxic antibiotics.

The alkylating agents can be nitrogen mustards, nitrosoureas, tetrazines, aziridines, cisplatins and derivatives. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. Further, the alkylating agents also include procarbazine and hexamethylmelamin.

The antimetabolites include anti-folates, fluoropyrimidines, deoxynucleoside analogues and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents include the vinca alkaloids and taxanes, Vinca alkaloids include vincristine, vinblastine, vinorelbine, vindesine, and vinflunine. Taxanes include paclitaxel, docetaxel

Podophyllotoxin is also an anti-microtubule agent and acts in a manner similar to that of vinca alkaloids.

Topoisomerase inhibitors include irinotecan and topotecan, etoposide, doxorubicin, mitoxantrone, teniposide, novobiocin, merbarone, and aclarubicin.

The cytotoxic antibiotics include anthracyclines, bleomycin, mitomycin C, and actinomycin. Important anthracyclines are doxorubicin, daunorubicin, epirubicin, idarubicin, pirarubicin, aclarubicin, and mitoxantrone. Immune checkpoint inhibitors include those that target CTLA4, PD-1, or PD-L1, and include Ipilimumab (targets CTLA-4), Nivolumab (targets PD-1), Pembrolizumab (targets PD-1), Atezolizumab (targets PDL-1), Avelumab (targets PDL-1), Durvalumab (targets PDL-1), and Cemiplimab (targets PD-1). Further, also those inhibitors that exhibit ubiquitin ligase actively, such as CISH (cytokine-inducible SH2-containing protein) and CBLB.

Cytokines useful in cancer therapy and hence in combination with the presently disclosed method of the 2^{nd} aspect of the invention include treatment with interleukin 2 (IL-2), interleukin 12 (IL-12), interleukin 15 (IL-15), interleukin 21 (IL-21), granulocyte-macrophage stimulating factor (GM-CSF), interferon-α (IFN-α), tumour necrosis factor (TNF-α), TGF-β, and CSF-1.

Targeted antibody therapies (which in the present context only relate to those that do not include checkpoint inhibitor treatments, since these as indicated above constitute a separate group of anti-cancer therapies) include therapy with those antibodies, antibody-drug conjugates, and other antibody-derived therapies that target various cancer-associated antigens. Targeted antibody therapies are for example those that target HER-2 (targeted by Pertuzumab, Trastuzumab, and Trastuzumab emtansine), VEGF (targeted by Bevacizumab), EGFR (targeted by Cetuximab, Necitumumab, and Panitumumab), CD38, disialoganglioside GD2 antigen (targeted by Dinutuximab), SLAMF7 (targeted by Elotuzumab), CD38 (targeted by Isatuximab), CCR4 (targeted by Mogamulizumab), CD20 (targeted by Obinutuzumab, Ofatumumab, Rituximab, Ibritumomab tiuxetan, and I¹³¹ tositumomab), VEGFR2 (targeted by Ramucirumab), CD33 (targeted by Gemtuzumab ozogamicin), CD30 (targeted by Brentuximab vedotin), CD22 (targeted by Inotuzumab ozogamicin and Moxetumomab pasudotox), CD79B (targeted by Polatuzumab vedotin), Nectin-4 (targeted by Enfortumab vedotin), TROP2 (targeted by Sacituzumab govitecan), and CD19/CD3 (targeted by Blinatumomab).

### Formulation and Administration of (Poly)Peptides and Vectors

In general, immunization with the peptides identified via the methods disclosed herein follow methods generally known in the art, both in terms of routes of administration, formulation technology, etc.

A composition comprising a peptide (or in some cases a vector encoding such a peptide) identified according to the invention thus typically contain an immunological adjuvant, which is commonly an aluminium based adjuvant or one of the other adjuvants described in the following:

Adjuvants to enhance effectiveness of an immunogenic composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE, although not required) formulated into submicron particles using a microfluidizer such as Model 110Y microfluidizer (Microfluidics, Newton, MA), (b) SAF, containing 10% Squalane, 0.4% Tween 80, 5% pluronic-blocked polymer L121, and thr-MDP, either microfluidized into a submicron emulsion or vortexed to generate a larger particle size emulsion, and (c) Ribi adjuvant system (RAS), (Ribi Immunochem, Hamilton, MT) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, MA) may be used or particles generated therefrom such as ISCOMs (immunostimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumour necrosis factor (TNF), etc.; and (6) other substances that act as immunostimulating agents to enhance the effectiveness of the composition.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (e.g. the immunising antigen or immunogen or polypeptide or protein or nucleic acid, pharmaceutically acceptable carrier (and/or diluent and/or vehicle), and adjuvant) typically will contain diluents, such as water, saline, glycerol, ethanol, etc. Additionally, auxiliary substances, such as wetting or emulsifying agents, pH buffering substances, and the like, may be present in such vehicles.

Pharmaceutical compositions can thus contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N. J. 1991).

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the relevant immunogen, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individuals to be treated (e.g. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount of immunogen will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (cf. eg. WO 98/20734). Additional formulations suitable for other modes of administration include oral, pulmonary and nasal formulations, suppositories, and transdermal applications. In the case of nucleic acid vaccination and antibody treatment, also the intravenous or intraarterial routes may be applicable.

Dosage treatment may be a single dose schedule or a multiple dose schedule, for instance in a prime-boost dosage regimen (a primary immunization followed by one or more booster immunizations) or in a burst regimen, i.e. sequential “primary” immunizations. The vaccine may be administered in conjunction with other immunoregulatory agents as may be convenient or desired.

When the precise composition and format of a vaccine has been determined as set forth above, the invention relies generally on methods well known to the medical practitioner for inducing immunity and follow up on patients. This also entails dosing of the vaccines (which in the case protein/peptide based vaccines typically entails administration of between 0.5 μg and 500 μg per dosage), typically provided as at least a priming dosage followed by one or several booster immunizations, cf. above.

Malignant neoplasms that can be targeted by the present invention can be selected from the group consisting of an epithelial tumour, a non-epithelial tumour, and a mixed tumour. The epithelial tumour may be both a carcinoma or an adenocarcinoma, and the non-epithelial tumour or mixed tumour is typically a liposarcoma, a fibrosarcoma, a chondrosarcoma, an osteosarcoma, a leiomyosarcoma, a rhabomyosarcoma, a glioma, a neuroblastoma, a medullablastoma, a malignant melanoma, a malignant meningioma, a neurofibrosarcoma, a leukemia, a myeloproleferative disorder, a lymphoma, a hemangiosarcoma, a Kaposi's sarcoma, a malignant teratoma, a dysgerminoma, a seminoma, or a choriosarcoma.

Also, the anatomic location of the malignant neoplasm can be anywhere in body; it may of the eye, the nose, the mouth, the tongue, the pharynx, the oesophagus, the stomach, the colon, the rectum, the bladder, the ureter, the urethra, the kidney, the liver, the pancreas, the thyroid gland, the adrenal gland, the breast, the skin, the central nervous system, the peripheral nervous system, the meninges, the vascular system, the testes, the ovaries, the uterus, the uterine cervix, the spleen, bone, or cartilage.

If the method of the 2^{nd} aspect is combined with other anti-cancer treatments, these are typically selected from immunotherapy using immune checkpoint inhibitors (ICIs)—such as based on PD-1/PDL-1, CTLA-4 mechanisms)-radiotherapy, surgery, chemotherapy, antibody therapy or various types of immunological cancer treatment, including other types of active specific immune therapy, adoptive cell-based immunotherapies (e.g. CAR-T cells, TCR-T cells, TILs, DC cells) and other approaches used in immuno-oncology.

In general, details pertaining to formulation and administration of the present immunogenic compositions enabled by the 1^{st} aspect can in preferred embodiments follow the same general principles as those which are disclosed in the context of immunization with neopeptides and neopeptide encoding vectors disclosed in WO 2020/141207, WO 2020/182901, WO 2021/123232, WO 2021/204911, PCT/EP2021/071380, and PCT/EP2021/069582, which are all included by reference herein in their entirety. In addition, the technologies for cancer treatment in each and any of these disclosures can be combined with the presently disclosed invention, meaning that e.g. a neoepitope/neopeptide-based immunization approach can be combined with an immunization strategy as disclosed herein

### Embodiments of the 3^{rd} Aspect of the Invention

The 3^{rd} aspect relates to a computer or computer system for identifying immunogenic amino acid sequences in a sample of malignant tissue from a patient, said computer or computer system comprising
1) one or more input component(s) for inputting amino acid sequence or mRNA data;
2) optionally executable code for determining amino acid sequences from mRNA data;
3) a database comprising amino acid sequences of expression products of genomic sequences;
4) executable code for identifying presence in inputted amino sequences or amino acid sequences encoded by inputted mRNA of sequences present in the amino acid sequences in the database;
5) executable code that identifies and optionally ranks amino acid sequences identified by the executable code in 4 in accordance with their predicted ability to bind a selection of MHC molecules; and
6) a component for outputting or storing the amino acid sequences identified and/or ranked wherein said genomic sequences in 3 constitute a subset of all sequences of the genome of a species and where said subset is constituted by sequences, which encode proteinaceous expression products in at most 5% of samples from any healthy tissue type found in the species, where said healthy tissue type optionally does not include testis tissue and/or brain tissue. Thus, the computer or computer systems of the invention is designed to implement the method of the 1^{st} aspect of the invention.

The input component is typically selected from any device for inputting data into a computer memory or storage medium: in principle, a simple keyboard connected to the computer can serve this purpose, but typically data will be read from an external data carrier or data source by a connected disk drive or other data carrier (a memory stick, memory card, network associated storage) or via a network or internet connection and a suitable protocol for file transfer (FTP, FTPS, SFTP, CSP, HTTP or HTTPS, AS2, 3-, and -4, or PeSIT). Likewise, storage (permanent or transitory) of sequences can be accomplished with any convenient data carrier or storage medium (a hard drive, a solid state hard drive, a memory stick) but also directly in the memory (RAM) of the computer or computer system. The storage format can be any convenient format such as in the form of records in a relation database (both row-oriented and column-oriented), an object database, but also as entries in text files (e.g. as comma separated values or a suitable XML format, or as a simple file system or other similar root-and-tree structure).

The output component is likewise any suitable output device, optionally coupled to a storage medium as described above. In addition to such storage media, output can be presented on paper via a printer or on a monitor. Also, the sequence data outputted can be later input into a device for peptide synthesis or—if the desired immunogen is a nucleic acid based vaccine—into a nucleic acid synthesizer.

The executable code(s) in the computer or computer system is capable of accessing the linked input devices and storage media as well as the computer working memory in order to perform the necessary operations of encoding amino acid sequences, sorting and comparing amino acid sequences etc.

Executable code for determining amino acid sequences from mRNA is straightforward to encode and is based on the genetic code, where triplets of nucleotides are translated in to amino acid residues.

### Embodiments of the 4^{th} Aspect of the Invention

The 4^{th} aspect relates to a method for determining whether a cancer patient is likely to benefit from an anti-cancer immunotherapy, comprising determining the number of EVEs (typically ERVs), which are expressed in said patient, and categorizing the cancer patient as being likely to benefit from the anti-cancer immunotherapy if said number of expressed EVEs/ERVs exceeds a predefined threshold.

As shown in Example 5, it turns out that a high burden of expressed ERVs in cancer patients correlate strongly with survival rates when these patients receive cancer immunotherapy. Notably, this increased survival is unrelated to the exact mode of immunotherapy and can be observed in both immune checkpoint inhibitor treated patients and in patients treated with adoptive T-cell therapy, i.e. in patients receiving passive as well as active cancer immunotherapy.

The predefined threshold of expressed ERVs is thereby typically at least 1.1 times the number of expressed ERVs in the average patient suffering from any cancer or from the specific type of cancer. This number may be higher (at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3, at least 3.5, at least 4, at least 4.5, at least 5, at least 7.5, and at least 10). However, depending on the exact cancer, the exact number may vary. Generally, the methods utilised in the present application's Example 5 can be used to determine the threshold for ERV expression, which in a given population having a certain cancer provides for a predictive effect. However, as one useful threshold, it is possible to consider eligible for the immunotherapy those patients, who exhibit at least 50 ERVs transcribed at a rate ≥1 RNA transcripts per million RNA transcripts from malignant tissue of the cancer patient. The number 50 is however not mandatory.

Generally, the threshold should be set to ensure that it would provide for a hazard ratio of at least 0.4 when applied to the combined dataset of transcripts derivable from Hugo et al. 2016 and Riaz et al. 2017 (see Example 5) and when computed using the Lifelines package (lifelines.readthedocs.io/en/stable/Citing%20lifelines.html) with the CoxPHFitter( ).fit command with default settings; see also Davidson-Pilon C (2019), DOI: 10.21105/joss.01317. This hence means that if the EVE burden is expressed as a specified number of EVE/ERV transcripts per million (TPM) or, alternatively, as the number of EVE/ERVs with TPM>1, then the specified number is above the threshold when it provides for a hazard ratio of at least 0.4 when applied to the dataset of Hugo et al. 2016 and Riaz et al. 2017.

As mentioned, the cancer immunotherapy is not limited to any specific immunotherapy approach and can hence be selected from therapeutic cancer vaccination, adoptive T-cell therapy (such as CAR-T cell therapy), targeted antibody therapy, immune checkpoint inhibitor therapy, and cytokine therapy. Any of the immune therapeutic approaches that are discussed as co-treatments under the 2^{nd} aspect of the invention above can be said cancer immunotherapy.

A particularly preferred embodiment of the 4^{th} aspect of the invention is one wherein the therapeutic cancer vaccination induces an immune response against cancer-associated antigens and/or cancer-specific antigens, in particular against neoantigens or antigens expressed from the genomic sequences as those genomic sequences that are specifically discussed in the embodiments of the 1^{st} aspect of the invention: the expressed genomic sequences are hence preferably selected from endogenous retroelement (EVE) sequences, such as ERV sequences, nuOFR sequences, and alternatively spliced sequences, but in essence any genomic sequences can be pre-selected if they are considered to be likely to be expressed under certain circumstances and if they contain the necessary MHC binding amino acid stretches. However, the preferred expressed genomic sequences to target are expressed ERV sequences.

Consequently, the cancer therapy is in preferred aspects the method disclosed in the 2^{nd} aspect of the invention and any embodiment thereof disclosed herein.

In a preferred embodiment of the 4^{th} aspect of the invention and the embodiments thereof discussed above, the cancer patient has a low tumour mutational burden; i.e. the cancer patient has a tumour mutational burden, which does not in itself correlate with clinical benefit of the cancer immunotherapy.

As is clear from the above, the finding that high ERV burden correlates with higher survival rates in patients treated with anti-cancer immunotherapy, even in patients that have a low tumour mutational burden, renders it feasible to subject such patients with high ERV burden to anti-cancer immunotherapy. In other words, when carrying out the method of the 4^{th} aspect, it is preferred that when categorizing the cancer patient as being likely to benefit from the anti-cancer immunotherapy, then the patients are subsequently subjected to the cancer immunotherapy discussed in detail above.

### EXAMPLE 1

### Identification of EVEs, Comprising hERVs, in Human Cancer Samples

A FASTA file containing the cDNA sequences of 33966 human EVEs, denoted hERVs in this example, was downloaded from the gEVE database (Nakagawa S and Takahashi MU. (2016)). The hERV FASTA file was appended to a FASTA file containing transcript cDNA sequences from annotated human genes. The human transcript cDNA file was downloaded from Ensembl (Yates A. D. et al. (2020)) along with the human reference genome. The combined cDNA fasta file containing annotated human genes and hERV sequences was index using Kallisto (Bray N. L. et al. (2020)).

RNA-seq data from 400 melanoma patients was downloaded from The Cancer Genome Atlas (TCGA) database (Cancer Genome Atlas Network (2015)). ERV expression was quantified using Kallisto with an hERV aware index, generated as explained above. Using a threshold of 1 TPM, between 21-901 hERVs were identified in each of the 400 melanoma samples. Furthermore, using a threshold value of 1 TPM for hERV expression, it is observed that the vast majority of ERVs are expressed in a small subset of the tumour samples. This highlights the potential for a personalized approach for selecting the best suitable target in any given tumour. Importantly, a subset of the ERVs have not been observed to be expressed in healthy tissue to any significant degree.

### EXAMPLE 2

### Selection of ERV-Derived Epitopes from the CT26 Mouse Tumour Cell Line

A GTF file containing a list of 61,184 mouse EVEs, denoted mERVs in in this example, and their genomic locations was downloaded from the gEVE database (Nakagawa S and

Takahashi MU. (2016)). The mERV GTF file was appended to a gene annotation GTF file containing information about annotated mouse transcripts. The mouse reference transcript GTF file was downloaded from Ensembl (Yates A. D. et al. (2020)) along with the mouse reference genome. The reference genome and the reference transcript+mERV GTF file was used to create indexes for STAR (Dobin A. et al. (2013)) and RSEM (Li B. et al. (2011)).

Previously generated RNA-seq data from the CT26 cell line (using a standard poly-A selection library preparation protocol and sequenced on an Illumina sequencing machine) was used to detect mERVs. The reads were mapped using STAR and RSEM with mERV aware indexes, generated as described above. 3484 expressed ERVs were identified using this pipeline. The 10 highest expressed mERVs are shown in Table 1.

TABLE 1
Top 10 expressed mERVs identified from RNA-seq
data from the CT26 tumour cell line

| ERV ID | Length | TPM |
|---|---|---|
| Mmus38.chr8.123426364.123428661.− | 2298 | 1213.61 |
| Mmus38.chr8.123428313.123431900.− | 3588 | 665.73 |
| Mmus38.chr6.120132388.120134214.− | 1827 | 374.65 |
| Mmus38.chr8.123431904.123433835.− | 1932 | 249.06 |
| Mmus38.chr6.120128961.120131645.− | 2685 | 210.63 |
| Mmus38.chr6.120131612.120132532.− | 921 | 104.77 |
| Mmus38.chr8.121839776.121840789.+ | 1014 | 59.10 |
| Mmus38.chr13.75644704.75645216.− | 513 | 50.42 |
| Mmus38.chr1.64965118.64966917.− | 1800 | 44.07 |
| Mmus38.chr8.121838853.121839896.+ | 1044 | 43.32 |

MHC class I and-II ligands were identified from all expressed mERVs using MHC ligand prediction tools: netMHCpan-4.1 (pubmed.ncbi.nlm.nih.gov/32406916/) for MHC Class I and netMHCIIpan-4.0 (pubmed.ncbi.nlm.nih.gov/32406916/). The MHC ligand prediction tools utilize amino acid sequence information as well as transcript expression levels to generate an integrated MHC ligand probability score. Ligand predictions were only generated for the mouse MHCs present in the CT26 cell line (H2-Dd, H2-Kd, H2-Ld and H2-IAd).

The optimum epitope encoding sequences were subsequently identified from each mERV. Overlapping 27-mers spaced with 1 amino acid were generated from each ERV. For each 27-mer an overall MHC ligand score was calculated using the MHC ligand predictions, cf. above. The best scoring MHC-I and MHC-II ligands, (defined as the ligands with the highest probability scores) were identified and the final combined MHC ligand probability score was calculated using the following equation:

$${P}_{MHC} = 1 - \sqrt{( 1 - {P}_{MHCI} ) * ( 1 - {P}_{MHCII} )}$$

where PMHC, PMHCI, and PMHCII are the probability scores for MHC binding in general, binding to MHC Class I, and binding to MHC Class II, respectively.

All overlapping 27-mers were ranked by their MHC ligand probability and the best 27-mer from each mERV was selected as the best epitope hotspot for that ERV. The 10 highest scoring epitope hotspots are listed in Table 2.

TABLE 2
Top 10 mERV epitope hotspots in CT26

| | | | | MHC-I | | MHC-II | |
|---|---|---|---|---|---|---|---|
| ERV ID | TPM | Epitope hotspot | Score | allele | MHC-I ligand | allele | MHC-II ligand |
| Mmus38.chr1. | 105.91 | IYEQATKECRAALAPK | 0.80 | H-2-Dd | IAPKKEQRL | H-2-IAd | TKECRAALAPKKEQR |
| 55778849. | | KEQRLTRLAQG | | | (SEQ ID NO: 281) | | (SEQ ID NO: 291) |
| 55780315.+ | | (SEQ ID NO: 271) | | | | | |
| Mmus38.chr16. | 59.19 | TYGIPAHNRQTLQSTE | 0.80 | H-2-Ld | IPAHNRQTL | H-2-IAd | RQTLQSTELQKSQQE |
| 90347358. | | LQKSQQEWISV | | | (SEQ ID NO: 282) | | (SEQ ID NO: 292) |
| 90349199.− | | (SEQ ID NO: 272) | | | | | |
| Mmus38.chr1. | 34.36 | MSIQRVPVEPIPSLPP | 0.80 | H-2-Ld | VPVEPIPSL | H-2-IAd | SIQRVPVEPIPSLPP |
| 112268369. | | GTMGLILGRGS | | | (SEQ ID NO: 283) | | (SEQ ID NO: 293) |
| 112269289.− | | (SEQ ID NO: 273) | | | | | |
| Mmus38.chr10. | 30.35 | AYQKIKQALLTAPALR | 0.79 | H-2-Kd | AYQKIKQAL | H-2-IAd | YQKIKQALLTAPALR |
| 5721414. | | LPDLTKPFELF | | | (SEQ ID NO: 284) | | (SEQ ID NO: 294) |
| 5723273.+ | | (SEQ ID NO: 274) | | | | | |
| Mmus38.chrY. | 27.69 | PQGSRLQGPPYAESLP | 0.79 | H-2-Dd | QGPPYAESL | H-2-IAd | GPPYAESLPCVVRQP |
| 85093497. | | CVVRQPCAERQ | | | (SEQ ID NO: 285) | | (SEQ ID NO: 295) |
| 85095335.− | | (SEQ ID NO: 275) | | | | | |
| Mmus38.chr1. | 69.28 | NSPSLQAHLQALQAVQ | 0.79 | H-2-Dd | NSPSLQAHL | H-2-IAd | SPSLQAHLQALQAVQ |
| 170944154. | | ERVWKPLAAAY | | | (SEQ ID NO: 286) | | (SEQ ID NO: 296) |
| 170947741.+ | | (SEQ ID NO: 276) | | | | | |
| Mmus38.chr7. | 61.16 | PQVLGTDNGPAFVSQV | 0.79 | H-2-Dd | NGPAFVSQV | H-2-IAd | GPAFVSQVSQSVAKL |
| 29617989. | | SQSVAKLLGID | | | (SEQ ID NO: 287) | | (SEQ ID NO: 297) |
| 29621588.+ | | (SEQ ID NO: 277) | | | | | |
| Mmus38.chr9. | 222.87 | FLYSKLPSKLIQSTSS | 0.79 | H-2-Kd | LYSKLPSKL | H-2-IAd | LPSKLIQSTSSTSPR |
| 7800731. | | TSPRSLTSEGL | | | (SEQ ID NO: 288) | | (SEQ ID NO: 298) |
| 7801702.− | | (SEQ ID NO: 278) | | | | | |
| Mmus38.chrx. | 31.77 | PRSQGPQRYGNRFVRT | 0.79 | H-2-Dd | QGPQRYGNRF | H-2-IAd | NRFVRTQEAAREATQ |
| 121443888. | | QEAAREATQED | | | (SEQ ID NO: 289) | | (SEQ ID NO: 299) |
| 121445672.− | | (SEQ ID NO: 279) | | | | | |
| Mmus38.chr6. | 38.88 | PNSSKLTVAVAQSDAA | 0.79 | H-2-Dd | SGPSAPPKI | H-2-IAd | SSKLTVAVAQSDAAG |
| 104225272.10 | | GKSGPSAPPKI | | | (SEQ ID NO: 290) | | (SEQ ID NO: 300) |
| 4226231.− | | (SEQ ID NO: 280) | | | | | |

### EXAMPLE 3

### Validation of mERV-Derived Ligands using MS Immunopeptidomics

FASTA files containing amino acid sequences of 61,184 mERVs were downloaded from the gEVE database (Nakagawa S and Takahashi MU. (2016)). These FASTA files were concatenated with the Uniprot (SwissProt and Trembl) [1 Aug. 2021] for the mouse proteome to create a search database for the mass spectrometry (MS) raw files obtained from LC-MS/MS analysis of CT26 cell line (IFNy+ and IFNy−) and tumour samples subjected to an immunoaffinity purification protocol as previously described (Purcell A et al. (2019)). Briefly, cell pellets or tissue from the cancer of interest were homogenized and lysed to release pMHC complexes which were subsequently captured in an immunoaffinity column using MHC-specific antibodies. MHC-bound peptides are separated from the MHC molecule, eluted and analysed by LC-MS/MS. As such, the MHC-bound peptides are separated by their mass-to-charge (m/z) ratio and their identity can be determined based on their fragmentation spectra.

To analyse the spectra, the software tool PEAKS (X-Pro) was employed allowing for a de novo assisted database search. The mouse-specific database described above was used for the search.

TABLE 3
Immunopeptidomics-identified mERV ligands found on untreated CT26 cell line, IFN-
treated CT26 cell line, and pooled CT26-derived tumours.

| | SEQ ID | | | | IFN | IFN | |
|---|---|---|---|---|---|---|---|
| pMHC ligand | NO: | ERV ID | −10lgP | TPM | neg | pos | Tumour |
| SPSYVYHQ | 1 | Mmus38_chr8_123426364_123428661_− | 40.05 | 1213.61 | 1 | 0 | 0 |
| TQQYHQLKTIG | 2 | Mmus38_chr8_123426364_123428661_− | 33.04 | 1213.61 | 1 | 0 | 0 |
| SMAKLRERL | 3 | Mmus38_chr8_123426364_123428661_− | 21.52 | 1213.61 | 1 | 0 | 1 |
| SGPPYYEGV | 4 | Mmus38_chr8_123426364_123428661_− | 41.08 | 1213.61 | 1 | 0 | 0 |
| VLTQQYHQL | 5 | Mmus38_chr8_123426364_123428661_− | 26.4 | 1213.61 | 1 | 1 | 1 |
| LVLTQQYHQLK | 6 | Mmus38_chr8_123426364_123428661_− | 37.7 | 1213.61 | 1 | 0 | 0 |
| VLTQQYHQLK | 7 | Mmus38_chr8_123426364_123428661_− | 38.7 | 1213.61 | 1 | 0 | 0 |
| WFTTLISTI | 8 | Mmus38_chr8_123426364_123428661_− | 22.07 | 1213.61 | 1 | 0 | 0 |
| LIILLLIL | 9 | Mmus38_chr8_123426364_123428661_− | 28.16 | 1213.61 | 1 | 0 | 0 |
| HGPSYWGL | 10 | Mmus38_chr8_123426364_123428661_− | 29.39 | 1213.61 | 1 | 0 | 1 |
| DYITVSNNL | 11 | Mmus38_chr8_123426364_123428661_− | 26.43 | 1213.61 | 1 | 0 | 0 |
| PSYVYHQF | 12 | Mmus38_chr8_123426364_123428661_− | 43.14 | 1213.61 | 1 | 1 | 1 |
| CFYADHTG | 13 | Mmus38_chr8_123426364_123428661_− | 31.38 | 1213.61 | 1 | 0 | 1 |
| AAPTGTTWA | 14 | Mmus38_chr8_123426364_123428661_− | 31.81 | 1213.61 | 1 | 1 | 0 |
| VLTQQYHQLKTIG | 15 | Mmus38_chr8_123426364_123428661_− | 38.07 | 1213.61 | 1 | 0 | 0 |
| LGGVNPVAL | 16 | Mmus38_chr8_123426364_123428661_− | 32.4 | 1213.61 | 1 | 1 | 0 |
| CCFYADHTGL | 17 | Mmus38_chr8_123426364_123428661_− | 21.7 | 1213.61 | 1 | 1 | 0 |
| CFYADHTGL | 18 | Mmus38_chr8_123426364_123428661_− | 43.39 | 1213.61 | 1 | 1 | 1 |
| FYADHTGL | 19 | Mmus38_chr8_123426364_123428661_− | 37.43 | 1213.61 | 1 | 1 | 1 |
| SPSYVYHQF | 20 | Mmus38_chr8_123426364_123428661_− | 53.96 | 1213.61 | 1 | 1 | 1 |
| TSAQRAELI | 21 | Mmus38_chr8_123428313_123431900_− | 32.42 | 665.73 | 1 | 1 | 1 |
| HPTVPNPYNLL | 22 | Mmus38_chr8_123428313_123431900_− | 49.75 | 665.73 | 1 | 1 | 1 |
| LPQGFKNSPTL | 23 | Mmus38_chr8_123428313_123431900_− | 43.25 | 665.73 | 1 | 1 | 0 |
| WYTDGSSFL | 24 | Mmus38_chr8_123428313_123431900_− | 37.87 | 665.73 | 1 | 1 | 1 |
| HPTVPNPYNL | 25 | Mmus38_chr8_123428313_123431900_− | 46.4 | 665.73 | 1 | 1 | 1 |
| YQKMKALL | 26 | Mmus38_chr8_123428313_123431900_− | 29.44 | 665.73 | 1 | 1 | 1 |
| YPLLGRDLL | 27 | Mmus38_chr8_123428313_123431900_− | 22.97 | 665.73 | 1 | 1 | 1 |
| AYQEIKQALL | 28 | Mmus38_chr8_123428313_123431900_− | 33.72 | 665.73 | 1 | 1 | 1 |
| HPTSQPLFAF | 29 | Mmus38_chr8_123428313_123431900_− | 25.17 | 665.73 | 1 | 1 | 0 |
| YQEIKQAL | 30 | Mmus38_chr8_123428313_123431900_− | 32.32 | 665.73 | 1 | 1 | 1 |
| GYQKMKALL | 31 | Mmus38_chr8_123428313_123431900_− | 45.6 | 665.73 | 1 | 1 | 1 |
| VGPKGQPL | 32 | Mmus38_chr8_123428313_123431900_− | 23.88 | 665.73 | 1 | 1 | 1 |
| AYQEIKQAL | 33 | Mmus38_chr8_123428313_123431900_− | 33.43 | 665.73 | 1 | 1 | 1 |
| PTVPNPYNL | 34 | Mmus38_chr8_123428313_123431900_− | 34.55 | 665.73 | 1 | 0 | 1 |
| TVPNPYNL | 35 | Mmus38_chr8_123428313_123431900_− | 30.43 | 665.73 | 1 | 1 | 1 |
| KPSPPPSEF | 36 | Mmus38_chr8_123431904_123433835_− | 28.79 | 249.06 | 1 | 1 | 1 |
| GLQNAGRSPTNLA | 37 | Mmus38_chr8_123431904_123433835_− | 23.35 | 249.06 | 1 | 0 | 0 |
| QVLSDNGGP | 38 | Mmus38_chr8_123431904_123433835_− | 18 | 249.06 | 1 | 0 | 0 |
| PLRLGGNGQLQ | 39 | Mmus38_chr8_123431904_123433835_− | 32.44 | 249.06 | 1 | 0 | 0 |
| SPDRFGLF | 40 | Mmus38_chr8_123431904_123433835_− | 26.05 | 249.06 | 1 | 0 | 1 |
| PPPYGGQGPSSSD | 41 | Mmus38_chr8_123431904_123433835_− | 26.29 | 249.06 | 1 | 0 | 0 |
| PQDGTFNL | 42 | Mmus38_chr8_123431904_123433835_− | 41.36 | 249.06 | 1 | 1 | 1 |
| TPYDPEDPG | 43 | Mmus38_chr8_123431904_123433835_− | 35.05 | 249.06 | 1 | 1 | 1 |
| WPQDGTFNL | 44 | Mmus38_chr8_123431904_123433835_− | 51.5 | 249.06 | 1 | 1 | 1 |
| PPAADSTTSRAF | 45 | Mmus38_chr8_123431904_123433835_− | 44.69 | 249.06 | 1 | 1 | 1 |
| GEEKQRVLLE | 46 | Mmus38_chr8_123431904_123433835_− | 26.4 | 249.06 | 1 | 0 | 0 |
| TNLAKVKGITQGP | 47 | Mmus38_chr8_123431904_123433835_− | 35.79 | 249.06 | 1 | 0 | 0 |
| SAPDIGRKLE | 48 | Mmus38_chr8_123431904_123433835_− | 21.22 | 249.06 | 1 | 0 | 0 |
| FYADHTGI | 49 | Mmus38_chr8_121839776_121840789_+ | 37.43 | 59.1 | 1 | 1 | 1 |
| SRTEADYYL | 50 | Mmus38_chr8_121839776_121840789_+ | 27.68 | 59.1 | 1 | 0 | 0 |
| SGPPYYKGI | 51 | Mmus38_chr8_121839776_121840789_+ | 42.97 | 59.1 | 1 | 1 | 1 |
| CCFYADHTGI | 52 | Mmus38_chr8_121839776_121840789_+ | 21.7 | 59.1 | 1 | 1 | 0 |
| CFYADHTGI | 53 | Mmus38_chr8_121839776_121840789_+ | 43.39 | 59.1 | 1 | 1 | 1 |
| KYFPANNKI | 54 | Mmus38_chr11_98069259_98069870_+ | 45.73 | 10.3 | 1 | 1 | 1 |
| QGPQIYGAL | 55 | Mmus38_chr11_98067757_98069424_+ | 31.29 | 10 | 1 | 1 | 1 |
| YYIPGLKGI | 56 | Mmus38_chr8_121835414_121838953_+ | 21.73 | 9.34 | 1 | 1 | 0 |
| DYLANIHHL | 57 | Mmus38_chr8_121835414_121838953_+ | 24.52 | 9.34 | 1 | 1 | 1 |
| HPNSQPLFAF | 58 | Mmus38_chr8_121835414_121838953_+ | 21.19 | 9.34 | 1 | 0 | 0 |
| TYVAGDTQV | 59 | Mmus38_chr8_121835414_121838953_+ | 38.78 | 9.34 | 1 | 1 | 0 |
| WPAEGTFYL | 60 | Mmus38_chr8_121833638_121835410_+ | 35.52 | 4.17 | 1 | 1 | 1 |
| FYLPTIRAV | 61 | Mmus38_chr8_121833638_121835410_+ | 26.7 | 4.17 | 1 | 1 | 1 |
| PAEGTFYL | 62 | Mmus38_chr8_121833638_121835410_+ | 31.32 | 4.17 | 1 | 1 | 0 |
| SYVPGGFDL | 63 | Mmus38_chr6_7725297_7727015_+ | 30.22 | 1.82 | 1 | 0 | 0 |
| SGPPYYEGI | 64 | Mmus38_chr6_7725297_7727015_+ | 34.37 | 1.82 | 1 | 1 | 0 |
| VGPNKVLI | 65 | Mmus38_chr13_68442584_68444656_− | 28.22 | 1.44 | 1 | 0 | 1 |
| RSPVYITHV | 66 | Mmus38_chr11_51666405_51669671_− | 30.72 | 0.44 | 1 | 1 | 0 |
| SSFGKLQYL | 67 | Mmus38_chr11_51666405_51669671_− | 25.8 | 0.44 | 1 | 0 | 0 |
| QSVVFPQI | 68 | Mmus38_chr11_51666405_51669671_− | 23.76 | 0.44 | 1 | 1 | 0 |
| SPVEAARNF | 69 | Mmus38_chr11_51666405_51669671_− | 33.34 | 0.44 | 1 | 1 | 1 |
| KYGSQNKYTGL | 70 | Mmus38_chr11_51666405_51669671_− | 45.98 | 0.44 | 1 | 0 | 0 |
| GYLISNQQV | 71 | Mmus38_chr16_91251454_91254057_+ | 27.26 | 0.12 | 1 | 0 | 0 |
| AIPKDFNP | 72 | Mmus38_chr9_25855705_25857714_− | 19.24 | 0.09 | 1 | 0 | 1 |
| SYQKMRALL | 73 | Mmus38_chr7_116433176_116436889_+ | 22.38 | 0.09 | 1 | 1 | 1 |
| LPDSGGPLI | 74 | Mmus38_chr7_116433176_116436889_+ | 44.39 | 0.09 | 1 | 1 | 1 |
| NGPAYTSQI | 75 | Mmus38_chr1_169543959_169547528_+ | 35.91 | 0.08 | 1 | 1 | 1 |
| GYQPGKVLA | 76 | Mmus38_chr17_34219868_34222795_+ | 32.09 | 0.06 | 1 | 1 | 1 |
| KYGTQNKYTGL | 77 | Mmus38_chr2_17613128_17616532_+ | 38.76 | 0.06 | 1 | 1 | 0 |
| APVEYLQI | 78 | Mmus38_chr17_34219868_34222795_+ | 22.76 | 0.06 | 1 | 0 | 0 |
| SPVEAAKNF | 79 | Mmus38_chr2_17613128_17616532_+ | 31.81 | 0.06 | 1 | 1 | 0 |
| AYTPGTTAV | 80 | Mmus38_chr2_181922040_181925600_+ | 29.95 | 0.04 | 1 | 0 | 1 |
| GPQESFSDF | 81 | Mmus38_chr3_156260400_156262304_− | 39.36 | 0.02 | 1 | 1 | 0 |
| AYAQISSTAI | 82 | Mmus38_chr2_87934671_87936479_+ | 30.75 | 0.02 | 1 | 0 | 0 |
| QGPPYAESL | 83 | Mmus38_chr2_87934671_87936479_+ | 32.75 | 0.02 | 1 | 1 | 0 |
| LGPPYYEGI | 84 | Mmus38_chr13_103716009_103720376_+ | 40.33 | 0.01 | 1 | 1 | 1 |
| ECCYADHTGI | 85 | Mmus38_chr9_12326759_12327097_− | 46.05 | 0 | 1 | 1 | 0 |
| AYNTGLTPCV | 86 | Mmus38_chrX_114133544_114135631_+ | 24.33 | 0 | 1 | 1 | 0 |
| DCCFYADHTGI | 87 | Mmus38_chrX_77934757_77935542_− | 49.24 | 0 | 1 | 1 | 0 |
| VPLIITPAI | 88 | Mmus38_chrX_9674213_9674725_− | 27.68 | 0 | 1 | 0 | 0 |
| KPSSSWDL | 89 | Mmus38_chrY_20670230_20671735_+ | 18.78 | 0 | 1 | 0 | 0 |
| YLASPAGTI | 90 | Mmus38_chrY_20670230_20671735_+ | 28.86 | 0 | 1 | 0 | 0 |
| LGPSTSTQI | 91 | Mmus38_chr5_145257238_145257882_− | 29.92 | 0 | 1 | 1 | 1 |
| LTEDPPAVRSTTF | 92 | Mmus38_chr13_21813594_21815231_+ | 36.94 | 0 | 1 | 0 | 0 |
| KGPANIFNKI | 93 | Mmus38_chr13_61516728_61519802_+ | 29.49 | 0 | 1 | 0 | 0 |
| LYLNIIKTI | 94 | Mmus38_chr17_36438521_36439126_+ | 26.31 | 0 | 1 | 1 | 1 |
| TPGDRIAQL | 95 | Mmus38_chr7_28232073_28233008_+ | 22.74 | 0 | 1 | 0 | 0 |
| LVDSGAQVS | 96 | Mmus38_chr6_86628075_86629190_+ | 21.12 | 0 | 1 | 0 | 0 |
| FYADHTGL | 97 | Mmus38_chr8_123426364_123428661_− | NaN | 1213.61 | 1 | 1 | 1 |
| SMAKLRERL | 98 | Mmus38_chr8_123426364_123428661_− | NaN | 1213.61 | 1 | 0 | 1 |
| QYHQLKTIG | 99 | Mmus38_chr8_123426364_123428661_− | NaN | 1213.61 | 0 | 0 | 1 |
| VLTQQYHQL | 100 | Mmus38_chr8_123426364_123428661_− | NaN | 1213.61 | 1 | 1 | 1 |
| SPHQVFNL | 101 | Mmus38_chr8_123426364_123428661_− | NaN | 1213.61 | 0 | 0 | 1 |
| SPSYVYHQF | 102 | Mmus38_chr8_123426364_123428661_− | NaN | 1213.61 | 1 | 1 | 1 |
| ISTTILDL | 103 | Mmus38_chr8_123426364_123428661_− | NaN | 1213.61 | 0 | 0 | 1 |
| MAKLRERL | 104 | Mmus38_chr8_123426364_123428661_− | NaN | 1213.61 | 0 | 0 | 1 |
| CFYADHTG | 105 | Mmus38_chr8_123426364_123428661_− | NaN | 1213.61 | 1 | 0 | 1 |
| CFYADHTGL | 106 | Mmus38_chr8_123426364_123428661_− | NaN | 1213.61 | 1 | 1 | 1 |
| PSYVYHQF | 107 | Mmus38_chr8_123426364_123428661_− | NaN | 1213.61 | 1 | 1 | 1 |
| IAAGVGTGTTAL | 108 | Mmus38_chr8_123426364_123428661_− | NaN | 1213.61 | 0 | 0 | 1 |
| HGPSYWGL | 109 | Mmus38_chr8_123426364_123428661_− | NaN | 1213.61 | 1 | 0 | 1 |
| GPCILNRL | 110 | Mmus38_chr8_123426364_123428661_− | NaN | 1213.61 | 0 | 0 | 1 |
| FGPCILNRL | 111 | Mmus38_chr8_123426364_123428661_− | NaN | 1213.61 | 0 | 1 | 1 |
| FGPCILNR | 112 | Mmus38_chr8_123426364_123428661_− | NaN | 1213.61 | 0 | 0 | 1 |
| LGPWRRPVAYL | 113 | Mmus38_chr8_123428313_123431900_− | NaN | 665.73 | 0 | 0 | 1 |
| GYQKMKALL | 114 | Mmus38_chr8_123428313_123431900_− | NaN | 665.73 | 1 | 1 | 1 |
| PTVPNPYNL | 115 | Mmus38_chr8_123428313_123431900_− | NaN | 665.73 | 1 | 0 | 1 |
| TVPNPYNL | 116 | Mmus38_chr8_123428313_123431900_− | NaN | 665.73 | 1 | 1 | 1 |
| TSAQRAELI | 117 | Mmus38_chr8_123428313_123431900_− | NaN | 665.73 | 1 | 1 | 1 |
| HPTVPNPYNLL | 118 | Mmus38_chr8_123428313_123431900_− | NaN | 665.73 | 1 | 1 | 1 |
| HPTVPNPYNL | 119 | Mmus38_chr8_123428313_123431900_− | NaN | 665.73 | 1 | 1 | 1 |
| TVPNPYNLL | 120 | Mmus38_chr8_123428313_123431900_− | NaN | 665.73 | 0 | 0 | 1 |
| VGPKGQPL | 121 | Mmus38_chr8_123428313_123431900_− | NaN | 665.73 | 1 | 1 | 1 |
| VPCQSPWNT | 122 | Mmus38_chr8_123428313_123431900_− | NaN | 665.73 | 0 | 0 | 1 |
| GYQKMKAL | 123 | Mmus38_chr8_123428313_123431900_− | NaN | 665.73 | 0 | 0 | 1 |
| WYTDGSSFL | 124 | Mmus38_chr8_123428313_123431900_− | NaN | 665.73 | 1 | 1 | 1 |
| YPLLGRDLL | 125 | Mmus38_chr8_123428313_123431900_− | NaN | 665.73 | 1 | 1 | 1 |
| AYQEIKQAL | 126 | Mmus38_chr8_123428313_123431900_− | NaN | 665.73 | 1 | 1 | 1 |
| YQEIKQAL | 127 | Mmus38_chr8_123428313_123431900_− | NaN | 665.73 | 1 | 1 | 1 |
| AWAETGGMGL | 128 | Mmus38_chr8_123428313_123431900_− | NaN | 665.73 | 0 | 0 | 1 |
| YQKMKALL | 129 | Mmus38_chr8_123428313_123431900_− | NaN | 665.73 | 1 | 1 | 1 |
| AYQEIKQALL | 130 | Mmus38_chr8_123428313_123431900_− | NaN | 665.73 | 1 | 1 | 1 |
| LYPALTPSI | 131 | Mmus38_chr8_123431904_123433835_− | NaN | 249.06 | 0 | 0 | 1 |
| TPYDPEDPG | 132 | Mmus38_chr8_123431904_123433835_− | NaN | 249.06 | 1 | 1 | 1 |
| PQDGTFNL | 133 | Mmus38_chr8_123431904_123433835_− | NaN | 249.06 | 1 | 1 | 1 |
| PAADSTTSRAF | 134 | Mmus38_chr8_123431904_123433835_− | NaN | 249.06 | 0 | 0 | 1 |
| KPSPPPSEF | 135 | Mmus38_chr8_123431904_123433835_− | NaN | 249.06 | 1 | 1 | 1 |
| SPDRFGLF | 136 | Mmus38_chr8_123431904_123433835_− | NaN | 249.06 | 1 | 0 | 1 |
| WPFSSSDL | 137 | Mmus38_chr8_123431904_123433835_− | NaN | 249.06 | 0 | 1 | 1 |
| WPQDGTFN | 138 | Mmus38_chr8_123431904_123433835_− | NaN | 249.06 | 0 | 0 | 1 |
| WPQDGTFNL | 139 | Mmus38_chr8_123431904_123433835_− | NaN | 249.06 | 1 | 1 | 1 |
| EDPGKLTAL | 140 | Mmus38_chr8_123431904_123433835_− | NaN | 249.06 | 0 | 0 | 1 |
| PPAADSTTSRAF | 141 | Mmus38_chr8_123431904_123433835_− | NaN | 249.06 | 1 | 1 | 1 |
| FGPCIINR | 142 | Mmus38_chr8_121839776_121840789_+ | NaN | 59.1 | 0 | 0 | 1 |
| CFYADHTGI | 143 | Mmus38_chr8_121839776_121840789_+ | NaN | 59.1 | 1 | 1 | 1 |
| FYADHTGI | 144 | Mmus38_chr8_121839776_121840789_+ | NaN | 59.1 | 1 | 1 | 1 |
| SGPPYYKGI | 145 | Mmus38_chr8_121839776_121840789_+ | NaN | 59.1 | 1 | 1 | 1 |
| PPIKDDLI | 146 | Mmus38_chr17_34998853_34999251_− | NaN | 15.22 | 0 | 0 | 1 |
| YFPANNKI | 147 | Mmus38_chr11_98069259_98069870_+ | NaN | 10.3 | 0 | 0 | 1 |
| KYFPANNKI | 148 | Mmus38_chr11_98069259_98069870_+ | NaN | 10.3 | 1 | 1 | 1 |
| QGPQIYGAL | 149 | Mmus38_chr11_98067757_98069424_+ | NaN | 10 | 1 | 1 | 1 |
| WTAVLPYAL | 150 | Mmus38_chr8_121835414_121838953_+ | NaN | 9.34 | 0 | 1 | 1 |
| DYLANIHHL | 151 | Mmus38_chr8_121835414_121838953_+ | NaN | 9.34 | 1 | 1 | 1 |
| WPAEGTFYL | 152 | Mmus38_chr8_121833638_121835410_+ | NaN | 4.17 | 1 | 1 | 1 |
| FYLPTIRAV | 153 | Mmus38_chr8_121833638_121835410_+ | NaN | 4.17 | 1 | 1 | 1 |
| LPDSGGPLI | 154 | Mmus38_chr4_146503839_146505761_+ | NaN | 2.56 | 1 | 1 | 1 |
| NPVITDQL | 155 | Mmus38_chr18_82699627_82701696_+ | NaN | 2.31 | 0 | 0 | 1 |
| SPVEAARNF | 156 | Mmus38_chr14_7589302_7591872_− | NaN | 0.22 | 1 | 1 | 1 |
| AYVANGKVV | 157 | Mmus38_chr14_7589302_7591872_− | NaN | 0.22 | 0 | 1 | 1 |
| GYQSCPTISSV | 158 | Mmus38_chr14_7589302_7591872_− | NaN | 0.22 | 0 | 0 | 1 |
| APVKYLQI | 159 | Mmus38_chr2_174984494_174986305_+ | NaN | 0.19 | 0 | 1 | 1 |
| HAPVKYLQI | 160 | Mmus38_chr2_174984494_174986305_+ | NaN | 0.19 | 0 | 0 | 1 |
| AYAQISST | 161 | Mmus38_chr2_174984494_174986305_+ | NaN | 0.19 | 0 | 0 | 1 |
| QGPQRYGNRF | 162 | Mmus38_chr2_174984494_174986305_+ | NaN | 0.19 | 0 | 0 | 1 |
| YQKMRALL | 163 | Mmus38_chr7_116433176_116436889_+ | NaN | 0.09 | 0 | 0 | 1 |
| SYQKMRALL | 164 | Mmus38_chr7_116433176_116436889_+ | NaN | 0.09 | 1 | 1 | 1 |
| NGPAYTSQI | 165 | Mmus38_chr1_169543959_169547528_+ | NaN | 0.08 | 1 | 1 | 1 |
| VGPNKVLI | 166 | Mmus38_chr1_85137432_85139519_− | NaN | 0.03 | 1 | 0 | 1 |
| WYLSDHTDL | 167 | Mmus38_chr1_85137432_85139519_− | NaN | 0.03 | 0 | 0 | 1 |
| PGPHRSQSL | 168 | Mmus38_chr13_103716009_103720376_+ | NaN | 0.01 | 0 | 0 | 1 |
| AIPKDFNP | 169 | Mmus38_chr13_103716009_103720376_+ | NaN | 0.01 | 1 | 0 | 1 |
| LGPPYYEGI | 170 | Mmus38_chr13_103716009_103720376_+ | NaN | 0.01 | 1 | 1 | 1 |
| LGPPYYEG | 171 | Mmus38_chr13_103716009_103720376_+ | NaN | 0.01 | 0 | 0 | 1 |
| GYQPGKVLA | 172 | Mmus38_chr4_143985403_143988237_− | NaN | 0.01 | 1 | 1 | 1 |
| METLISHL | 173 | Mmus38_chr9_15168036_15168659_+ | NaN | 0 | 0 | 0 | 1 |
| LGPTVPYTL | 174 | Mmus38_chr1_7887129_7888718_− | NaN | 0 | 0 | 0 | 1 |
| MGPHKITKLL | 175 | Mmus38_chr4_24822469_24823491_+ | NaN | 0 | 0 | 0 | 1 |
| AYQKIKQAL | 176 | Mmus38_chr5_122195673_122198165_+ | NaN | 0 | 0 | 0 | 1 |
| LGPSTSTQI | 177 | Mmus38_chr5_145257238_145257882_− | NaN | 0 | 1 | 1 | 1 |
| PAAAPPQGPI | 178 | Mmus38_chr7_103294893_103296881_+ | NaN | 0 | 0 | 0 | 1 |
| PAAAPPQGPIA | 179 | Mmus38_chrX_76894672_76896660_+ | NaN | 0 | 0 | 0 | 1 |
| RPKESPFEF | 180 | Mmus38_chr7_75779070_75780392_− | NaN | 0 | 0 | 0 | 1 |
| AYVPLTTTAL | 181 | Mmus38_chr7_75779070_75780392_− | NaN | 0 | 0 | 1 | 1 |
| AYVPLTTT | 182 | Mmus38_chr7_75779070_75780392_− | NaN | 0 | 0 | 0 | 1 |
| AYVPLTTTA | 183 | Mmus38_chr7_75779070_75780392_− | NaN | 0 | 0 | 1 | 1 |
| AYTPGTTAV | 184 | Mmus38_chr11_121313552_121317034_− | NaN | 0 | 1 | 0 | 1 |
| LYLNIIKTI | 185 | Mmus38_chr17_36438521_36439126_+ | NaN | 0 | 1 | 1 | 1 |
| PAAAPPQGPL | 186 | Mmus38_chr18_6352668_6354761_− | NaN | 0 | 0 | 0 | 1 |
| PAAAPPQGPLA | 187 | Mmus38_chr18_6352668_6354761_− | NaN | 0 | 0 | 0 | 1 |
| LTFGPCILNRL | 188 | Mmus38_chr16_28812840_28813082_+ | NaN | 0 | 0 | 1 | 1 |
| LSLPRSKLI | 189 | Mmus38_chr14_50984088_50984378_− | NaN | 0 | 0 | 0 | 1 |
| CCFYSDHTGI | 190 | Mmus38_chr13_3419954_3420313_− | NaN | 0 | 0 | 0 | 1 |
| SVSRTEAI | 191 | Mmus38_chrX_35726739_35727725_+ | NaN | 0 | 0 | 0 | 1 |
| VLTQQYHQL | 192 | Mmus38_chr8_123426364_123428661_− | NaN | 1213.61 | 1 | 1 | 1 |
| CCFYADHTGL | 193 | Mmus38_chr8_123426364_123428661_− | NaN | 1213.61 | 1 | 1 | 0 |
| AAPTGTTWA | 194 | Mmus38_chr8_123426364_123428661_− | NaN | 1213.61 | 1 | 1 | 0 |
| LIILLLILL | 195 | Mmus38_chr8_123426364_123428661_− | NaN | 1213.61 | 0 | 1 | 0 |
| ATQQFQQL | 196 | Mmus38_chr8_123426364_123428661_− | NaN | 1213.61 | 0 | 1 | 0 |
| FYADHTGL | 197 | Mmus38_chr8_123426364_123428661_− | NaN | 1213.61 | 1 | 1 | 1 |
| SPSYVYHQF | 198 | Mmus38_chr8_123426364_123428661_− | NaN | 1213.61 | 1 | 1 | 1 |
| FTTLISTI | 199 | Mmus38_chr8_123426364_123428661_− | NaN | 1213.61 | 0 | 1 | 0 |
| FGPCILNRL | 200 | Mmus38_chr8_123426364_123428661_− | NaN | 1213.61 | 0 | 1 | 1 |
| LGGVNPVAL | 201 | Mmus38_chr8_123426364_123428661_− | NaN | 1213.61 | 1 | 1 | 0 |
| KSPWFTTL | 202 | Mmus38_chr8_123426364_123428661_− | NaN | 1213.61 | 0 | 1 | 0 |
| PSYVYHQF | 203 | Mmus38_chr8_123426364_123428661_− | NaN | 1213.61 | 1 | 1 | 1 |
| CFYADHTGL | 204 | Mmus38_chr8_123426364_123428661_− | NaN | 1213.61 | 1 | 1 | 1 |
| VGPKGQPL | 205 | Mmus38_chr8_123428313_123431900_− | NaN | 665.73 | 1 | 1 | 1 |
| TVPNPYNL | 206 | Mmus38_chr8_123428313_123431900_− | NaN | 665.73 | 1 | 1 | 1 |
| AYQEIKQALL | 207 | Mmus38_chr8_123428313_123431900_− | NaN | 665.73 | 1 | 1 | 1 |
| GYQKMKALL | 208 | Mmus38_chr8_123428313_123431900_− | NaN | 665.73 | 1 | 1 | 1 |
| AYQEIKQAL | 209 | Mmus38_chr8_123428313_123431900_− | NaN | 665.73 | 1 | 1 | 1 |
| YQEIKQAL | 210 | Mmus38_chr8_123428313_123431900_− | NaN | 665.73 | 1 | 1 | 1 |
| HPTSQPLFAF | 211 | Mmus38_chr8_123428313_123431900_− | NaN | 665.73 | 1 | 1 | 0 |
| YQKMKALL | 212 | Mmus38_chr8_123428313_123431900_− | NaN | 665.73 | 1 | 1 | 1 |
| LPQGFKNSPTL | 213 | Mmus38_chr8_123428313_123431900_− | NaN | 665.73 | 1 | 1 | 0 |
| WYTDGSSFL | 214 | Mmus38_chr8_123428313_123431900_− | NaN | 665.73 | 1 | 1 | 1 |
| TSAQRAELI | 215 | Mmus38_chr8_123428313_123431900_− | NaN | 665.73 | 1 | 1 | 1 |
| HPTVPNPYNLL | 216 | Mmus38_chr8_123428313_123431900_− | NaN | 665.73 | 1 | 1 | 1 |
| HPTVPNPYNL | 217 | Mmus38_chr8_123428313_123431900_− | NaN | 665.73 | 1 | 1 | 1 |
| YPLLGRDLL | 218 | Mmus38_chr8_123428313_123431900_− | NaN | 665.73 | 1 | 1 | 1 |
| PPAADSTTSRAF | 219 | Mmus38_chr8_123431904_123433835_− | NaN | 249.06 | 1 | 1 | 1 |
| TPYDPEDPG | 220 | Mmus38_chr8_123431904_123433835_− | NaN | 249.06 | 1 | 1 | 1 |
| KPSPPPSEF | 221 | Mmus38_chr8_123431904_123433835_− | NaN | 249.06 | 1 | 1 | 1 |
| WPQDGTFNL | 222 | Mmus38_chr8_123431904_123433835_− | NaN | 249.06 | 1 | 1 | 1 |
| WPFSSSDL | 223 | Mmus38_chr8_123431904_123433835_− | NaN | 249.06 | 0 | 1 | 1 |
| PQDGTFNL | 224 | Mmus38_chr8_123431904_123433835_− | NaN | 249.06 | 1 | 1 | 1 |
| CCFYADHTGI | 225 | Mmus38_chr8_121839776_121840789_+ | NaN | 59.1 | 1 | 1 | 0 |
| SGPPYYKGI | 226 | Mmus38_chr8_121839776_121840789_+ | NaN | 59.1 | 1 | 1 | 1 |
| FYADHTGI | 227 | Mmus38_chr8_121839776_121840789_+ | NaN | 59.1 | 1 | 1 | 1 |
| CFYADHTGI | 228 | Mmus38_chr8_121839776_121840789_+ | NaN | 59.1 | 1 | 1 | 1 |
| KYFPANNKI | 229 | Mmus38_chr11_98069259_98069870_+ | NaN | 10.3 | 1 | 1 | 1 |
| QGPQIYGAL | 230 | Mmus38_chr11_98067757_98069424_+ | NaN | 10 | 1 | 1 | 1 |
| TYVAGDTQV | 231 | Mmus38_chr8_121835414_121838953_+ | NaN | 9.34 | 1 | 1 | 0 |
| DYLANIHHL | 232 | Mmus38_chr8_121835414_121838953_+ | NaN | 9.34 | 1 | 1 | 1 |
| WTAVLPYAL | 233 | Mmus38_chr8_121835414_121838953_+ | NaN | 9.34 | 0 | 1 | 1 |
| YYIPGLKGI | 234 | Mmus38_chr8_121835414_121838953_+ | NaN | 9.34 | 1 | 1 | 0 |
| FYLPTIRAV | 235 | Mmus38_chr8_121833638_121835410_+ | NaN | 4.17 | 1 | 1 | 1 |
| PAEGTFYL | 236 | Mmus38_chr8_121833638_121835410_+ | NaN | 4.17 | 1 | 1 | 0 |
| WPAEGTFYL | 237 | Mmus38_chr8_121833638_121835410_+ | NaN | 4.17 | 1 | 1 | 1 |
| LPDSGGPLI | 238 | Mmus38_chr4_146503839_146505761_+ | NaN | 2.56 | 1 | 1 | 1 |
| SGPPYYEGI | 239 | Mmus38_chr6_7725297_7727015_+ | NaN | 1.82 | 1 | 1 | 0 |
| IPAGGAPLL | 240 | Mmus38_chr2_176257180_176258139_+ | NaN | 0.39 | 0 | 1 | 0 |
| APVKYLQI | 241 | Mmus38_chr2_174984494_174986305_+ | NaN | 0.19 | 0 | 1 | 1 |
| GPQESFSDF | 242 | Mmus38_chr2_174984494_174986305_+ | NaN | 0.19 | 1 | 1 | 0 |
| KYLQIKEIAE | 243 | Mmus38_chr2_174984494_174986305_+ | NaN | 0.19 | 0 | 1 | 0 |
| KYGTQNKYTGL | 244 | Mmus38_chr2_174984494_174986305_+ | NaN | 0.19 | 1 | 1 | 0 |
| SYQKMRALL | 245 | Mmus38_chr5_25233524_25236934_+ | NaN | 0.17 | 1 | 1 | 1 |
| FHNNFHVTAETL | 246 | Mmus38_chr1_169543959_169547528_+ | NaN | 0.08 | 0 | 1 | 0 |
| RSPVYITHV | 247 | Mmus38_chr1_169543959_169547528_+ | NaN | 0.08 | 1 | 1 | 0 |
| SPVEAARNF | 248 | Mmus38_chr1_169543959_169547528_+ | NaN | 0.08 | 1 | 1 | 1 |
| AYVANGKVV | 249 | Mmus38_chr1_169543959_169547528_+ | NaN | 0.08 | 0 | 1 | 1 |
| NGPAYTSQI | 250 | Mmus38_chr1_169543959_169547528_+ | NaN | 0.08 | 1 | 1 | 1 |
| QSVVFPQI | 251 | Mmus38_chr1_169543959_169547528_+ | NaN | 0.08 | 1 | 1 | 0 |
| SPVEAAKNF | 252 | Mmus38_chr2_17613128_17616532_+ | NaN | 0.06 | 1 | 1 | 0 |
| KYLQIKELAE | 253 | Mmus38_chr18_17938786_17940741_+ | NaN | 0.05 | 0 | 1 | 0 |
| QGPPYAESL | 254 | Mmus38_chr2_87934671_87936479_+ | NaN | 0.02 | 1 | 1 | 0 |
| GYQPGKVLA | 255 | Mmus38_chr2_87934671_87936479_+ | NaN | 0.02 | 1 | 1 | 1 |
| LGPPYYEGI | 256 | Mmus38_chr13_103716009_103720376_+ | NaN | 0.01 | 1 | 1 | 1 |
| ECCYADHTGI | 257 | Mmus38_chr9_12326759_12327097_− | NaN | 0 | 1 | 1 | 0 |
| WCFYADHTGI | 258 | Mmus38_chrX_45236369_45238402_− | NaN | 0 | 0 | 1 | 0 |
| NPTCGGKVDF | 259 | Mmus38_chrX_45236369_45238402_− | NaN | 0 | 0 | 1 | 0 |
| KPGVVAHAF | 260 | Mmus38_chrX_57733998_57734564_− | NaN | 0 | 0 | 1 | 0 |
| DCCFYADHTGI | 261 | Mmus38_chrX_77934757_77935542_− | NaN | 0 | 1 | 1 | 0 |
| AYVPLTTTA | 262 | Mmus38_chr7_75779070_75780392_− | NaN | 0 | 0 | 1 | 1 |
| AYVPLTTTAL | 263 | Mmus38_chr7_75779070_75780392_− | NaN | 0 | 0 | 1 | 1 |
| AYNTGLTPCV | 264 | Mmus38_chr7_13174342_13176462_+ | NaN | 0 | 1 | 1 | 0 |
| LYLNIIKTI | 265 | Mmus38_chr15_21304920_21305402_− | NaN | 0 | 1 | 1 | 1 |
| LTFGPCILNRL | 266 | Mmus38_chr16_28812840_28813082_+ | NaN | 0 | 0 | 1 | 1 |
| SCFYADHTGI | 267 | Mmus38_chr12_81431100_81431519_+ | NaN | 0 | 0 | 1 | 0 |
| AALQNPLTL | 268 | Mmus38_chr12_20226796_20228865_− | NaN | 0 | 0 | 1 | 0 |
| LGPSTSTQI | 269 | Mmus38_chr5_145257238_145257882_− | NaN | 0 | 1 | 1 | 1 |
| LGPPYYDGI | 270 | Mmus38_chr1_52318946_52320427_+ | NaN | 0 | 0 | 1 | 0 |

### EXAMPLE 4

### Defining an Exemplary List hERVs for Application in Immunotherapy

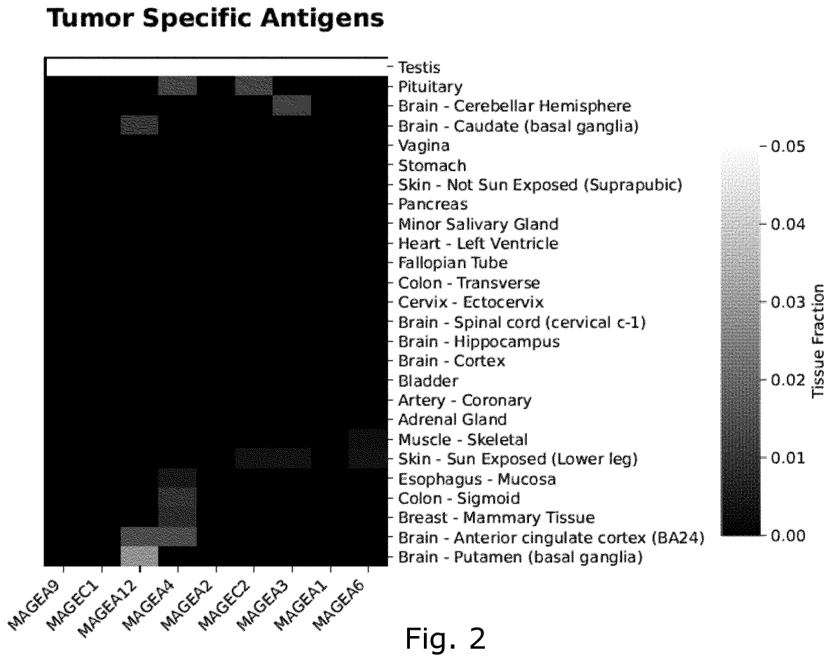

RNA-seq data of 6,125 healthy tissue samples from 51 different body sites were retrieved from the GTEx database (www.nature.com/articles/ng.2653) and analyzed for expression of hERVs and the MAGE family of tumour specific antigens (i.e. MAGEA1, MAGEA2, MAGEA3, MAGEA4, MAGEA6, MAGEA9, MAGEA10, MAGEA12, MAGEC1, and MAGEC2). The expression levels were quantified using Kallisto as described in Example 1. A transcript was considered expressed in a tissue sample if it occurred as ≥1 TPM. For each body site and each transcript, the fraction of samples that support the expression was calculated. The result is presented as a heatmap in FIG. 2.

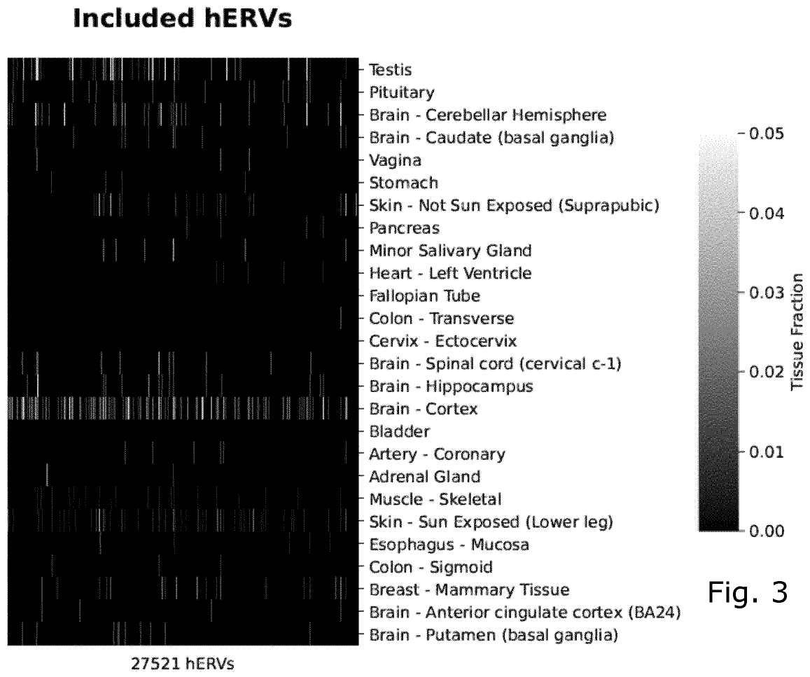

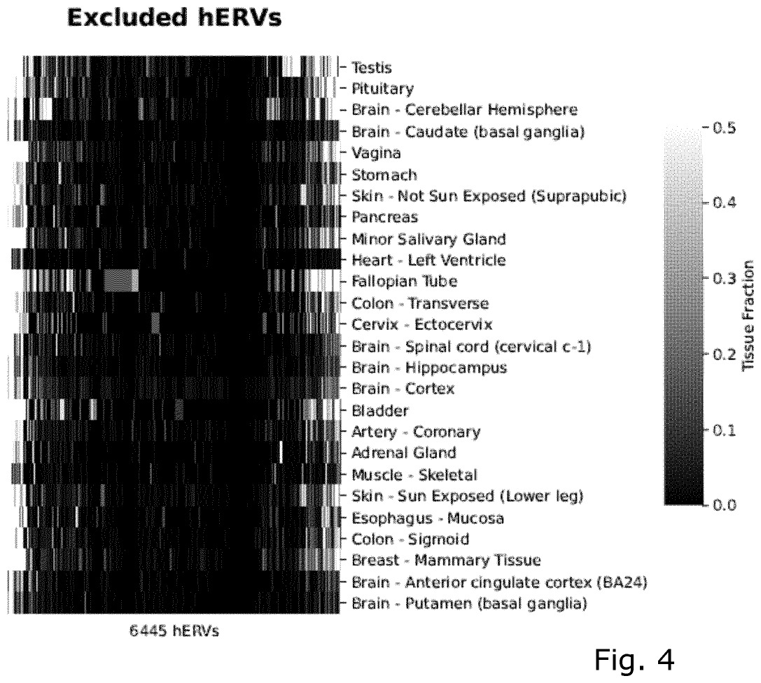

A matrix of the fraction of tissue samples that support expression of the respective hERV targets is presented in FIG. 3 as a heatmap. Similarly, the fraction of tissue samples that support expression of the deselected hERVs is shown in FIG. 4.

It is of importance to note that the selected hERV targets are very rarely observed as expressed in healthy tissue (evidenced by a “dark” coloured heatmap), while the deselected hERVs are observed to be expressed more frequently in at least one tissue (lighter coloured cells in the heatmap). This can be compared to FIG. 2, which presents the tissue expression of the TAAs discussed above, and which presents a tissue expression pattern between those of FIGS. 3 and 4.

The complete set of hERVs that was selected is constituted by 15,252 hERVs—these include the 12,202 hERVs for which the heatmap is shown in FIG. 3, but also includes those from the heatmap in FIG. 4, where the expression could be found in at most 5% of samples from a tissue different from brain and testis. The selection of such hERVS is occasioned by the immune privileged nature of brain and testis tissue.

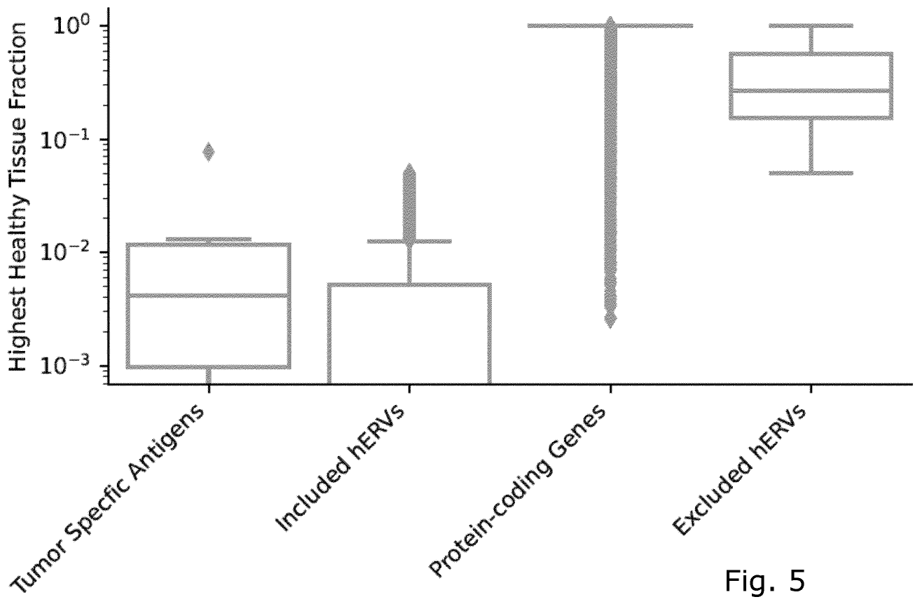

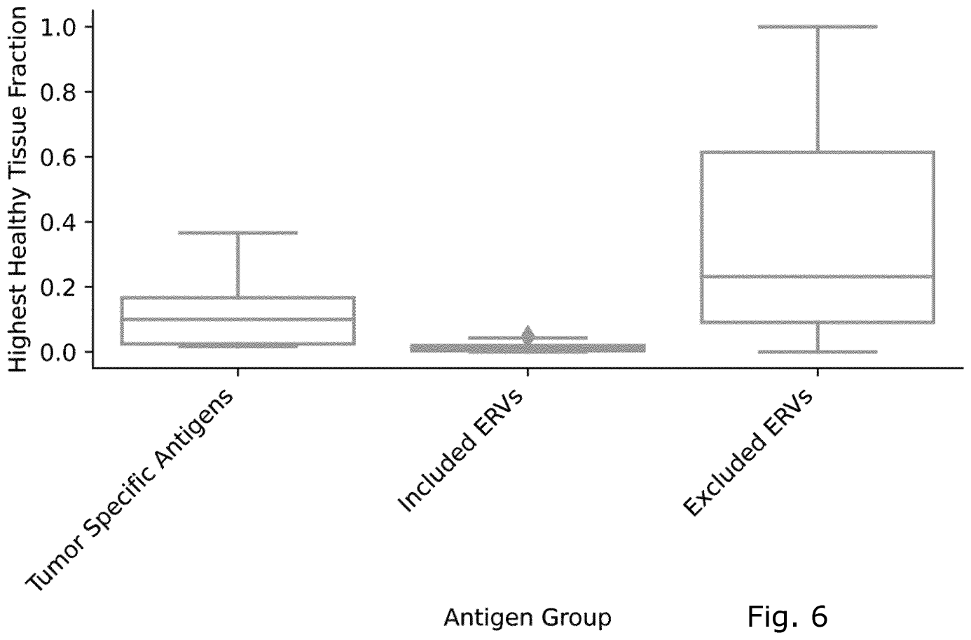

FIGS. 5 and 6 present the data in a different format, where the Y-axis indicates the highest healthy tissue fraction (values from 0 to 1) of 3 groups of antigens: TAAs, selected hERVs and excluded hERVs. FIG. 5 shows the fractions of exrpressed hERVs when the selected hERVs correspond to those of FIG. 3 (i.e. expression in brain and testis must also meet the 5% maximum threshold), and FIG. 6 shows the fractions when the selected hERVs allow for tissue expression in testis and/or brain. For the TSAs, expression in testis and brain is allowed in both figures.

The total of 27,521 hERVs found useful and safe for immune therapy in this example are provided in the table below.

In the name of the hERV sequences below the periods separate the following pieces of information:

Hsap38.X1.X2.X3.X4, where X1 is the human chromosome number, X2 is the start position, X2 is the end position, and X4 indicates the position on the + or − strand.

So, as an example “Hsap38.chr1.100599636.100600481.-” is located on human chromosome 1 (“chr1”), starts at position 100599636, ends at position 100600481, and is located on the minus strand−.

Tissue expression is indicated as −/−, +/−, −/+, and +/+, indicating “no tissue expression”, “expression in brain”, “expression in testis”, and “expression in brain and testis”, respectively.

Columns are 1: name, 2: tissue expression:

Lengthy table referenced here
US20250054576A1-20250213-T00001
Please refer to the end of the specification for access instructions.

### EXAMPLE 5

### Patient Stratification and ERV Burden

A high number of somatic mutations (known as the “tumour mutational burden”, abbreviated TMB) is predictive of better outcome for cancer patients receiving immunotherapy (cf. Hugo et al. 2016, Lauss et al. 2017, and Riaz et al. 2017).

On this background, it was investigated by the present inventors whether the number of expressed ERVs or, more generally, EVEs as a potential alternative source of neoantigens also would stratify patients receiving immunotherapy into groups with differential outcomes. In this context, the number of expressed ERVs/EVEs is in the following termed the “ERV burden” and “EVE burden”, respectively.

Three published studies of metastatic melanoma patients (stage III/IV) with baseline biopsies characterized by RNA sequencing and whole-exome sequencing were investigated: Hugo et al. 2016, Lauss et al. 2017, and Riaz et al. 2017.

### ERV/EVE Burden

The expression of endogenous retroviruses (using gEVE annotation) was computed by mapping RNA sequence reads with STAR (Dobin et al. 2017) and quantified by RSEM (Li B. and Dewey C. N. 2011). A threshold of 1 transcript per million (TPM) was used to define whether an ERV was considered expressed, and the ERV burden was determined as the number of ERVs/EVEs with an expression level above 1 TPM. For practical reasons, a high ERV/EVE burden was in this case defined as more than 50 ERVs or EVEs expressed in the tumour biopsy while less than 50 ERVs/EVEs were considered a low ERV/EVE burden. See below for a more general way to defined ERV/EVE burden as high vs. low.

### Tumour Mutational Burden

For the Hugo et al. (2016) and Lauss et al. (2017) studies, missense somatic mutations were identified by mapping whole-exome sequencing data from tumour biopsies and matched healthy samples to the human reference genome (GRCh38) with bwa mem (Li H (2017)). Somatic mutations were called using mutect2 (Benjamin D et al. 2019) and filtered using the GATK suite (Mckenna A et al. 2010). Somatic variants were annotated using the Variant Effect Predictor (McLaren W et al 2016). The tumour mutational burden (TMB) was defined as the number of missense somatic mutations. A TMB above 1000 was considered a high mutational burden, while a TMB less than 1000 missense somatic mutations was considered a low mutational burden.

For the Riaz et al. (2017) study, the high/low TMB group assignments were retrieved from the supplementary information due to data access restrictions of the whole-exome sequencing data.

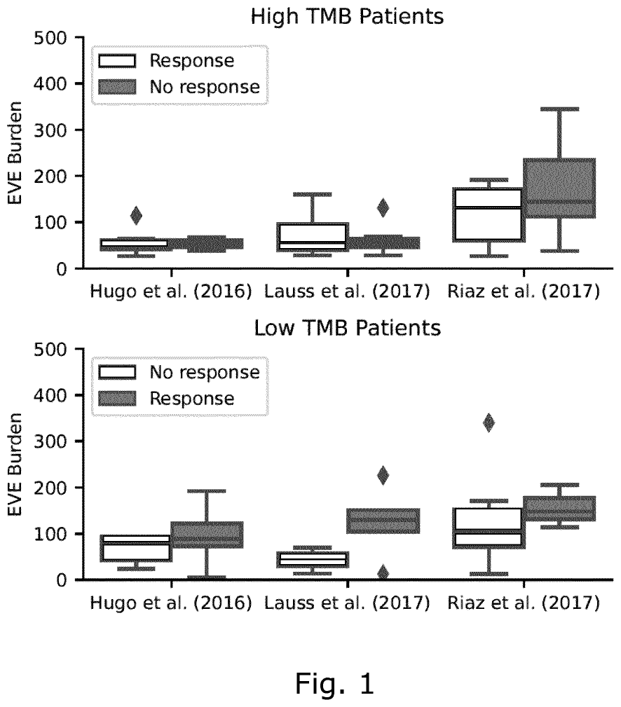

Data are shown in FIG. 1, which demonstrates that in the Low TMB group, responders are generally characterized by a higher EVE burden than non-responders.

### Patient Stratification

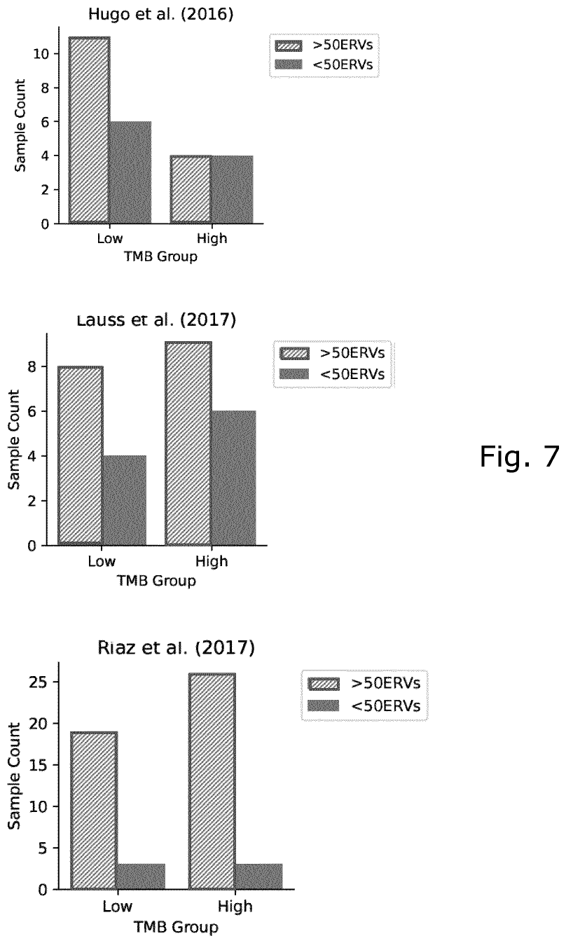

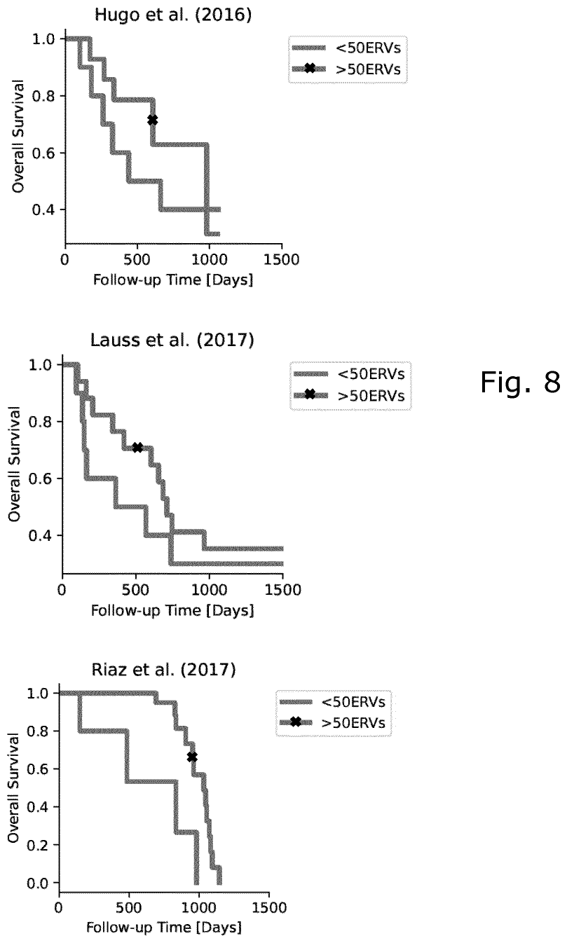

FIG. 7 shows the number of samples found in each patient group. Considering the ERV burden in isolation, it is observed that it can stratify patients based on their overall survival (cf. FIG. 8).

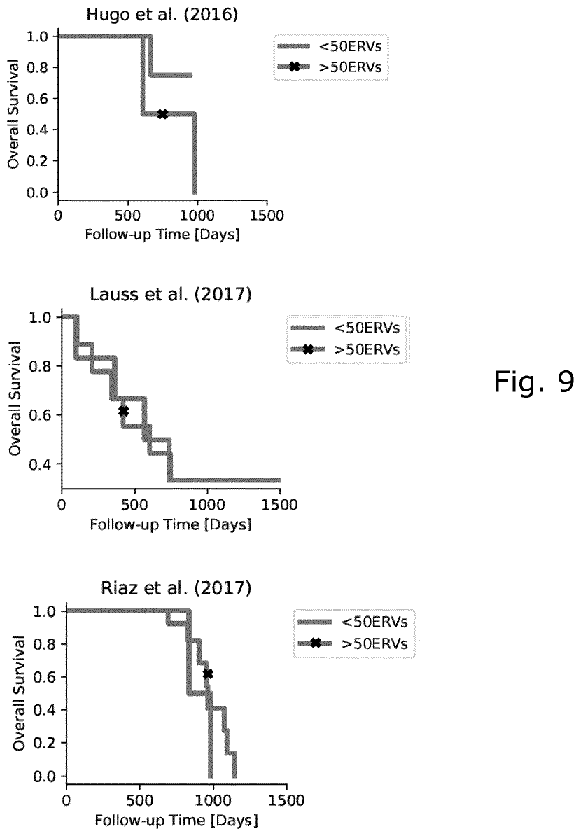

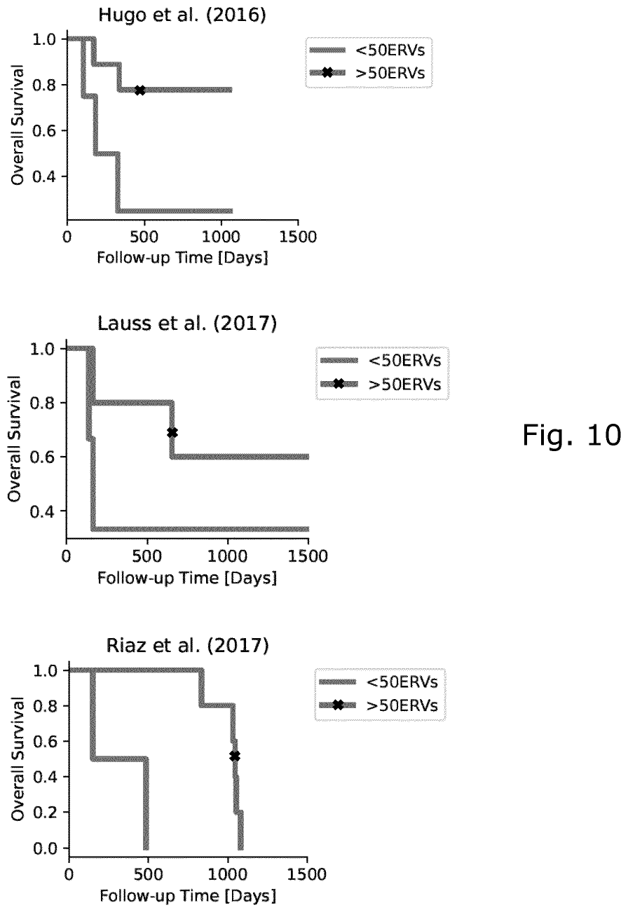

A further breakdown of the groups based on TMB reveals that for the high TMB group, the ERV burden is not predictive of patient survival (cf. FIG. 9), whereas an enhanced stratification signal is observed based on the ERV burden for the low TMB group (cf. FIG. 10). This corresponds to the data shown in FIG. 1.

This indeed suggests that the outcome of immunotherapy is dependent on the number of targetable tumour antigens, and that ERVs serve as an alternative source of tumour antigens that can be targeted.

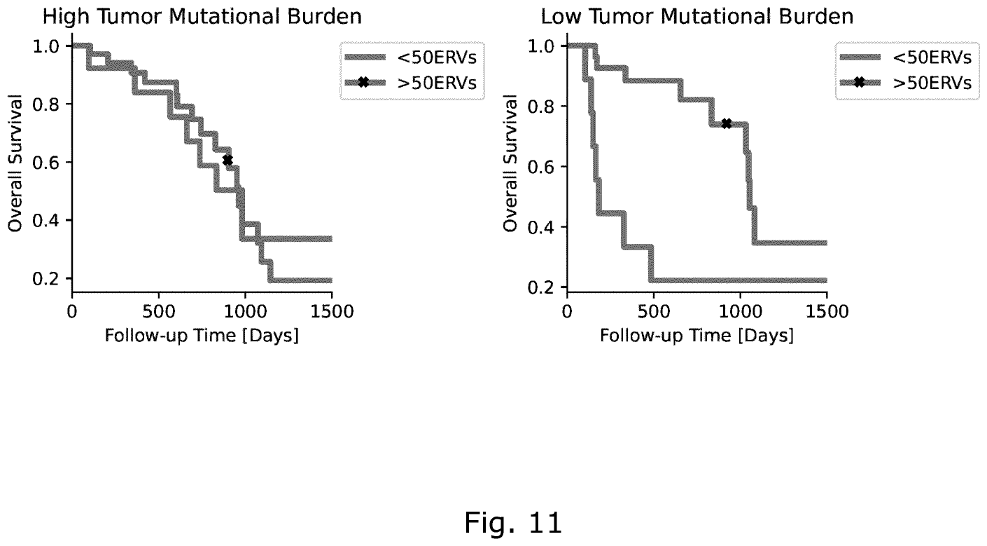

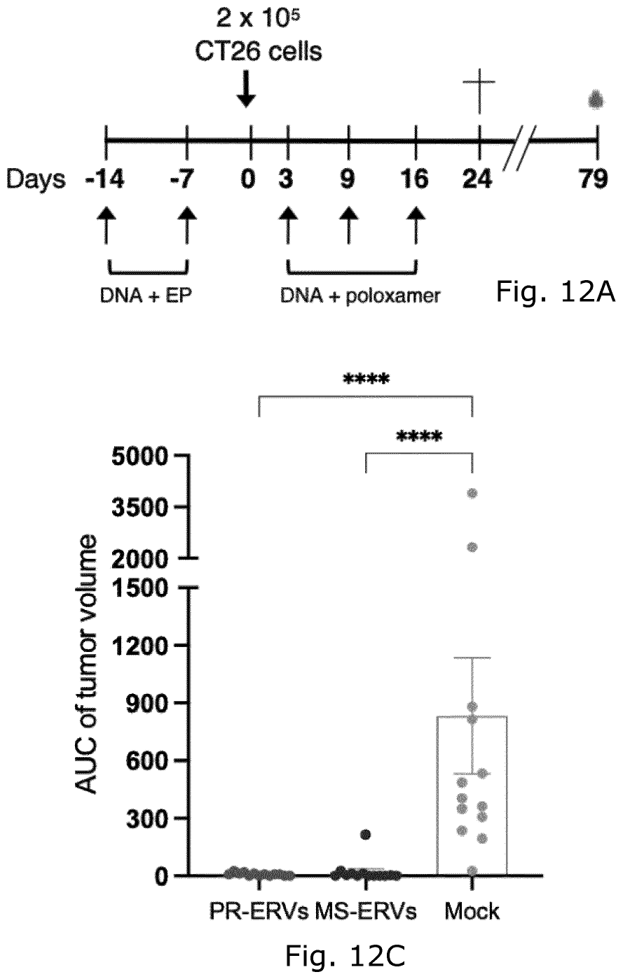

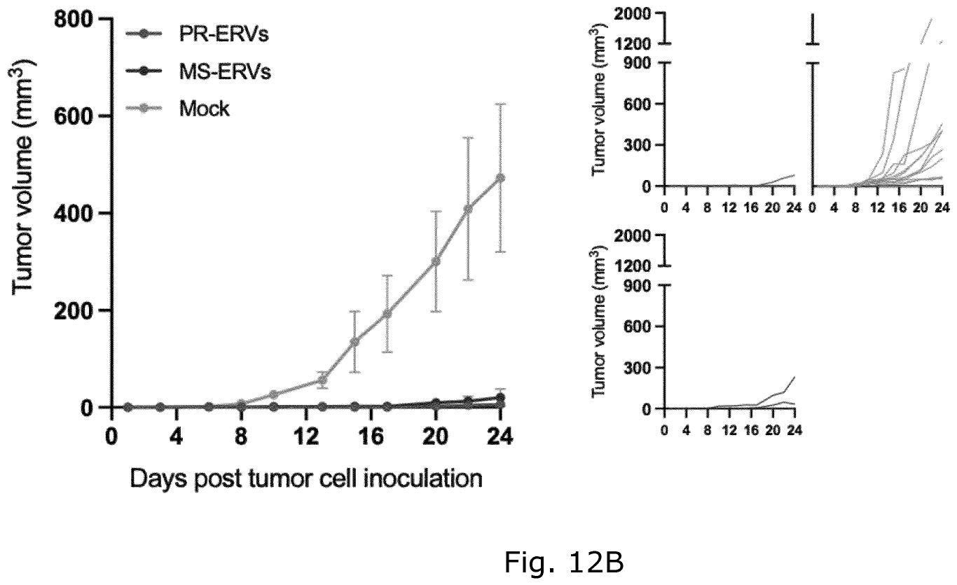

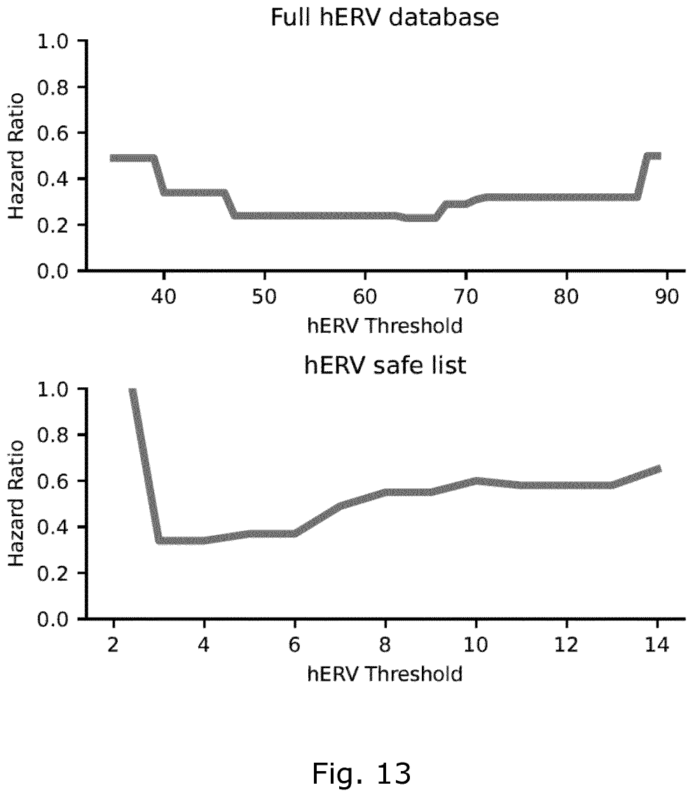

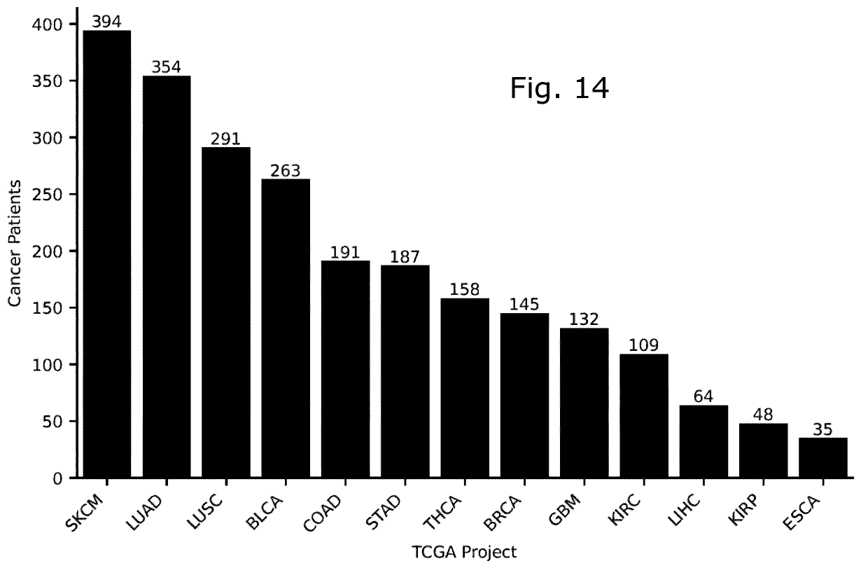

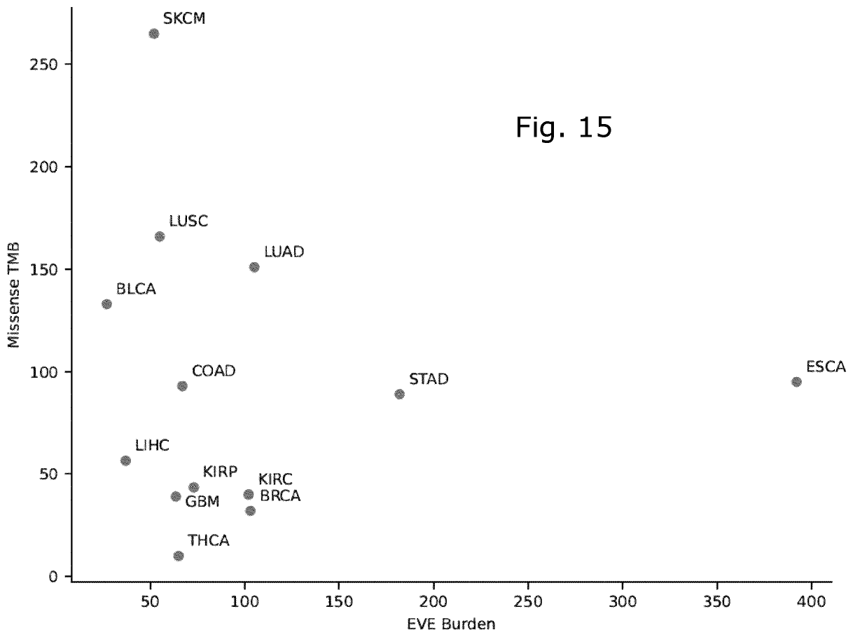

When collapsing all three studies a strong patient stratification signal based on the ERV burden for patients with low TMB across three types of immunotherapies was observed, while the ERV burden stratification signal could not be observed in patients with high TMB (FIG. 11).

In addition to ERVs serving as a new prognostic biomarker, the present analysis also supports the use of ERVs as complementary tumour antigen targets in personalized immunotherapy. ERVs could thereby constitute a tumour antigen source that enables the design of personalized immunotherapies for patients found to have a low tumour mutational burden and for cancer indications that generally are characterized by few somatic mutations.

To provide a robust measure of this patient stratification, the data was subset to the public data available in Hugo et al. 2016, Riaz et al. 2017 to define a universal scale that allows determination of whether an ERV/EVE burden is considered to be above or below the desired threshold.

An analysis for the EVE burden threshold for stratification was conducted on the full dataset and the list of hERVs that were found to be rarely expressed in healthy tissue (the list of ERVs provided in the table in Example 4) for the low-TMB patients. See FIG. 13. Choosing a threshold of hazard ratio=0.4, then the EVE burden thresholds for the full list span from 40 through to 87. While the reduced safe list has EVE burden thresholds from 3 through 6. Thus, low ERV patients can be considered to be those that stratify in the specified combined dataset from Hugo et al. 2016 and Riaz et al. 2017 with a hazard ratio below 0.4. Stratification is computed using the method described in Davidson-Pilon C (2019), with the CoxPHFitter( ).fit command with default settings.

An extensive number of cancer samples from TCGA was analysed to get an overview of which cancer types might be useful for targeting. The number and types of analysed samples is depicted in FIG. 14, while a scatterplot showing the project-wise median of the number of expressed EVEs (median EVE burden) vs the median number of missense somatic variants is depicted in FIG. 15.

### EXAMPLE 6

### Personalized ERV-Based Immunotherapy Induces Strong Anti-Tumour Effect and T-Cell Responses in the CT26 Colon Carcinoma Model

It was investigated whether ERVs can serve as relevant therapeutic cancer targets for personalized immunotherapy using the preclinical cancer model, CT26. ERV expression levels in in vivo grown CT26 tumours were quantified using RNA sequencing and BALB/c mice were subjected to an in silico designed immunotherapy for CT26 based on the ERV expression levels and the BALB/c MHC type. The in silico design comprised the 13 top ranked ERV peptides (PR-ERVs) encoded into a pTVG4 plasmid DNA (pDNA). For comparison, a pDNA construct was constructed to encode 13 ERV-derived peptides containing MHC-I ligands validated by immunopeptidomics (MS-ERVs).

The two pDNA constructs were administered in vivo through electroporation (EP) and in formulation with a nonionic block co-polymer (from here on: “poloxamer”) to increase the longevity of the pDNA after injection and thereby increase antigen expression and exposure.

To test whether ERVs can be relevant targets for immunotherapy, groups of mice were vaccinated intramuscularly (i.m.) with PR-ERVs, MS-ERVs or mock pDNA in one-week intervals and in a vaccine administration scheme comprising two EP-based prime immunizations followed by three poloxamer-based ones (FIG. 12A). Immunizations commenced two weeks prior to subcutaneous (s.c.) challenge with a tumourigenic dose of CT26 cancer cells. In contrast to mock pDNA-treated mice that developed tumours of significant end volume (FIG. 12B), mice vaccinated with PR-ERVs and MS-ERVs demonstrated strong prevention of CT26 tumour establishment (FIGS. 12B and 12C).

### Methods and Materials

### Mouse Cell Lines

The BALB/c syngeneic colon cancer cell line CT26 (#CRL2638) was purchased from ATCC and cultured in R10 medium prepared from RPMI (Gibco #72400-021) supplemented with 10% heat inactivated fetal calf serum (FCS, Gibco #10500-064) at 37° C. and 5% CO₂ as per supplier's instructions. Cells were grown to 60-70% confluency, treated with trypsin and washed 2× in serum free RPMI in preparation for inoculation in mice.

### Animal Studies

Animals were maintained at the animal facility at Evaxion Biotech, Hørsholm, Denmark. All experiments were conducted under license 2017-15-0201-01209 from the Danish Animal Experimentation Inspectorate in accordance with the Danish Animal Experimentation Act (BEK nr. 12 of Jul. 1, 2016), which is compliant with European Directive (2010/63/EU).

6-8 week old female BALB/cJRj SPF mice were acquired from Janvier Labs (France). The mice were acclimatized for one week before initiation of experiments. Mice from the different experimental groups (13 mice per group) were distributed across different cages to avoid potential cage effects. Mice were vaccinated weekly in left and right tibialis anterior muscles (i.m.) with 100 μg of research-grade DNA for a total of five immunizations. Vaccination commenced two weeks prior to subcutaneous CT26 cell inoculation (defined as study day 0). In the first two immunizations, 2×50 μl vaccine comprising DNA formulated in PBS was administered using Electroporation (EP). In the last three immunizations, DNA was formulated with block co-polymer poloxamer 188 (gifted by BASF, Germany) to a final concentration of 3% in PBS and administered in 2×75 μl vaccine solution. At the day of primary CT26 cell inoculation, 2×flank of the mice. For the tumour rechallenge experiment, mice that rejected primary cancer cell challenge and age-matched naïve mice were inoculated s.c. with the same tumourigenic dose of CT26 cells in the opposite (left) flank.

Upon establishment, tumours were measured three times a week using a digital calliper and tumour volumes, V, were calculated using the following formula:

$$V = π * {( {d}_{1} * {d}_{2} )}^{\frac{3}{2}}$$

where d₁ and d₂ are the orthogonal diameters of the tumour. Mice were euthanized through cervical dislocation when the majority of tumours in the control groups reached the maximum allowed size of 15 mm diameter in either direction or upon reaching humane endpoints.

GraphPad Prism 9 for Mac OS X was used for graphing, statistical analyses, and tools. Data were subjected to Kolmogorov-Smirnov test for normality (alpha=0.05). Parametric data were analysed by ordinary ANOVA with Sidak's multiple comparison correction. Non-parametric data were analysed by Mann-Whitney test (if two comparisons) or Kruskal-Wallis test with Dunn's multiple comparison correction (if more than two comparisons). For all results, the following levels of statistical significance are applied: *p<0.05, **p<0.01, ***p<0.001, ****p<0.0001.

### LIST OF REFERENCES

1) Balada, E. et al. (2009). Reviews in Medical Virology, 19(5), 273-286. doi.org/10.1002/rmv.622
2) Benjamin, D. et al. (2019). bioRxiv doi.org/10.1101/861054
3) Bronte, V. et al. (2003). Journal of Immunology (Baltimore, Md.: 1950), 171(12), 6396-6405. doi.org/10.4049/jimmunol.171.12.6396
4) Casares, N. et al. (2001). European Journal of Immunology, 31(6), 1780-1789. doi.org/10.1002/1521-4141(200106)31:6<1780::aid-immu1780>3.0.co;2-i
5) Chiappinelli, K. B. et al. (2015). Cell, 162(5), 974-986. doi.org/10.1016/j.cell.2015.07.011
6) Davidson-Pilon C (2019), Journal of Open Source Software, 4(40): 1-3 DOI: 10.21105/joss.01317
7) Dobin, A. et al. (2013). Bioinformatics, 29(1), 15-21. doi.org/10.1093/bioinformatics/bts635
8) Dupressoir, A. et al. (2012). Placenta, 33(9), 663-671. doi.org/10.1016/j.placenta.2012.05.005
9) Hugo, W. et al. (2016). Cell, 165, 35-44. dx.doi.org/10.1016/j.cell.2016.02.065
10) Hummel, J. et al. (2015). European Journal of Immunology, 45(6), 1748-1759. doi.org/10.1002/eji.201445366
11) Iramaneerat, K. et al. (2011). International Journal of Gynecological Cancer: Official Journal of the International Gynecological Cancer Society, 21(1), 51-57. doi.org/10.1097/IGC.0b013e3182021c1a
12) Kassiotis, G. (2014). Journal of Immunology (Baltimore, Md.: 1950), 192(4), 1343-1349. doi.org/10.4049/jimmunol.1302972
13) Kassiotis, G., and Stoye, J. P. (2016). Nature Reviews Immunology, 16(4), 207-219. doi.org/10.1038/nri.2016.27
14) Kershaw, M. H. et al. (2001). Cancer Research, 61(21), 7920-7924.
15) Kraus, B., et al. (2013). PloS One, 8(8), e72756. doi.org/10.1371/journal.pone.0072756
16) Kraus, B. et al. (2014). Virology Journal, 11, 58. doi.org/10.1186/1743-422X-11-58
17) Krishnamurthy, J. et al. (2015). Clinical Cancer Research: An Official Journal of the American Association for Cancer Research, 21(14), 3241-3251. doi.org/10.1158/1078-0432.CCR-14-3197
18) Lauss, M et al. (2017). Nature Communications, 8(1):1738 doi: 10.1038/s41467-017-01460-0
19) Li, B. and Dewey C. N. (2011). BMC Bioinformatics, 12;323 www.biomedcentral.com/1471-2105/12/323
20) Li, H. (2013). arXiv: 1303.3997v2 DOI: 10.6084/M9.FIGSHARE.963153.V1
21) Li, M., et al. (2017). Clinical Cancer Research: An Official Journal of the American Association for Cancer Research, 23(19), 5892-5911. doi.org/10.1158/1078-0432.CCR-17-0001
22) Lokossou, A. G. et al. (2020). Biology of Reproduction, 102(1), 185-198. doi.org/10.1093/biolre/ioz124
23) Magiorkinis, G. et al. (2012). Proceedings of the National Academy of Sciences of the United States of America, 109(19), 7385-7390. doi.org/10.1073/pnas.1200913109
24) Mangeney, M. (2005) et al. Cancer Research, 65(7), 2588-2591. doi.org/10.1158/0008-5472.CAN-04-4231
25) Mckenna, A. et al. (2010). Genome Res, 20(9), 1297-1303 doi: 10.1101/gr.107524.110
26) McLaren, W. et al. (2016). Genome Biology, 17:122 DOI: 10.1186/s13059-016-0974-4
27) Morozov, V. A. et al. (2013). PloS One, 8(8), e70399. doi.org/10.1371/journal.pone.0070399
28) Neukirch, L. et al. (2019). Oncotarget, 10(14), 1458-1472. doi.org/10.18632/oncotarget.26680
29) Pavlicek, A. and Jurka, J. (2006). In: Genomic Disorders: The Genomic Basis of Disease (pp. 57-72). Humana Press. doi.org/10.1007/978-1-59745-039-3_4
30) Peltonen, K. et al. (2021). In: Cancers 13(14), 3408. doi.org/10.3390/cancers13143408
31) Qian, C. et al. (2020). Vaccines, 8(1). doi.org/10.3390/vaccines8010139
32) Riaz, N. et al. (2017). Cell 171(4): 934-949. doi: 10.1016/j.cell.2017.09.028
33) Rice, J., et al. (2002). Journal of Immunology (Baltimore, Md.: 1950), 169(7), 3908-3913. doi.org/10.4049/jimmunol.169.7.3908
34) Roulois, D. et al. (2015). Cell, 162(5), 961-973. doi.org/10.1016/j.cell.2015.07.056
35) Saini, S. K. et al. (2020). Nature Communications, 11(1), 5660. doi.org/10.1038/s41467-020-19464-8
36) Scheeren, R. A. et al. (1992). Clinical and Experimental Immunology, 89(1), 94-99. doi.org/10.1111/j.1365-2249.1992.tb06884.x
37) Szpakowski, S., et al. (2009). Gene, 448(2), 151-167. doi.org/10.1016/j.gene.2009.08.006
38) Takeda, J. et al. (2000). Cellular Immunology, 204(1), 11-18. doi.org/10.1006/cimm.2000.1691
39) Vergara Bermejo, A. et al. (2020). International Journal of Molecular Sciences, 21(14), 4843. doi.org/10.3390/ijms21144843
40) Volkman, H. E. and Stetson, D. B. (2014). Nature Immunology, 15(5), 415-422. doi.org/10.1038/ni.2872
41) Wang-Johanning, F. et al. (2007). International Journal of Cancer, 120(1), 81-90. doi.org/10.1002/ijc.22256
42) Young, G. R. et al. (2014). Retrovirology, 11(1), 59. doi.org/10.1186/1742-4690-11-59
43) Zhou, F. et al. (2016). Oncotarget, 7(51), 84093-84117. doi.org/10.18632/oncotarget.11455
44) Nakagawa S and Takahashi MU. (2016). Database: the journal of biological databases and curation. 2016: baw087. Available from: academic.oup.com/database/article-lookup/doi/10.1093/database/baw087
45) Yates A. D. et al. (2020). Nucleic acids research, 48(D1): D682-8. Available from: academic.oup.com/nar/advance-article/doi/10.1093/nar/gkz966/5613682
46) Bray N. L. et al. (2020). Nature biotechnology 34(5): 525-7. Available from: www.ncbi.nlm.nih.gov/pubmed/27043002
47) Cancer Genome Atlas Network (2015). Genomic Classification of Cutaneous Melanoma. Cell. 18;161(7):1681-96. Available from: linkinghub.elsevier.com/retrieve/pii/S0092867415006340
48) Dobin A. et al. (2013). Bioinformatics. 29(1):15-21.
49) Li B. et al. (2011). BMC bioinformatics. 12(1):323. Available from: bmcbioinformatics.biomedcentral.com/articles/10.1186/1471-2105-12-323
50) Purcell et al. (2019). Nature Protocols. 14:1687-1707.

LENGTHY TABLES
The patent application contains a lengthy table section. A copy of the table is available in electronic form from the USPTO web site (https://seqdata.uspto.gov/?pageRequest=docDetail&DocID=US20250054576A1). An electronic copy of the table will also be available from the USPTO upon request and payment of the fee set forth in 37 CFR 1.19(b)(3).

1. A method for identifying immunogenic amino acid sequences in a sample of malignant tissue from a patient comprising
A) determining amino acid sequences of proteinaceous expression products from the malignant tissue,
B) analysing said amino acid sequences to identify therein proteinaceous expression products of selected genomic sequences in the patient's species,
C) identifying—in the proteinaceous expression products—the amino acid sequences, which are those that will bind to MHC molecules of the patient,
where said selected genomic sequences constitute a subset of all sequences of the genome of said species and where said subset is constituted by sequences, which encode proteinaceous expression products in at most 5% of samples from any healthy tissue, where said healthy tissue is a tissue of a type found in the patient, and where said healthy tissue optionally does not include testis tissue and/or brain tissue.

2. The method according to claim 1, wherein the healthy tissue does not include testis tissue; brain tissue; or testis and brain tissue.

3. The method according to claim 1, wherein the healthy tissue includes testis and brain tissue.

4. The method according to claim 1, wherein the genomic sequences are selected from endogenous viral elements (EVEs), such as endogenous retrovirus (ERV) sequences; novel or unannotated open reading frame (nuOFR) sequences; and genomic sequences that are transcribed as alternatively spliced sequences.

5. The method according to claim 4, wherein the genomic sequences are ERV sequences, in particular human ERV sequences.

6. The method according to claim 1, wherein the amino acid sequences of peptides that will bind to MHC molecules of the patient are amino acid sequences that will
bind both MHC Class I and MHC Class II molecules of the patient;
bind MHC Class I molecules, but not MHC Class II molecules of the patient; or
bind MHC Class II molecules, but not MHC Class I molecules of the patient.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled).

29. The method according to claim 1, wherein the amino acid sequences of the peptides that will bind to MHC molecules of the patient are presented by the MHC molecules on the surface of antigen presenting cells.

30. The method according to claim 1, wherein step A comprises determination of the amino acid sequence from mRNA of the patient's malignant tissue.

31. The method according to claim 1, wherein the selected genomic sequences are identified by determining the expression profile of genomic sequences across a plurality of samples from a plurality of tissues to select those genomic sequences that are expressed in <5% of the plurality of samples.

32. The method according to claim 31, wherein the plurality of samples of a plurality of tissues does not include samples from testis and brain tissue.

33. The method according to claim 31, wherein the plurality of samples of a plurality of tissues includes samples from testis and brain tissue.

34. A method of treating a malignant neoplasm in a patient, preferably a human patient, the method comprising sampling malignant tissue from the patient and identifying immunogenic amino acid sequences in the sample comprising
A) determining amino acid sequences of proteinaceous expression products from the malignant tissue,
B) analysing said amino acid sequences to identify therein proteinaceous expression products of selected genomic sequences in the patient's species,
C) identifying, in the proteinaceous expression products, the amino acid sequences, which are those that will bind to MHC molecules of the patient,
where said selected genomic sequences constitute a subset of all sequences of the genome of said species and where said subset is constituted by sequences, which encode proteinaceous expression products in at most 5% of samples from any healthy tissue, where said healthy tissue is a tissue of a type found in the patient, and where said healthy tissue optionally does not include testis tissue and/or brain tissue,
and subsequently administering to the patient one or more peptides identified in step C or one or more polypeptides comprising 2 or more peptides identified in step C, or one or more expression vectors encoding and capable of expressing said one or more peptides identified in step C or said one or more polypeptides comprising 2 or more peptides identified in step C so as to induce a specific adaptive immune response against said one or more peptides.

35. The method according to claim 34, wherein the healthy tissue does not include testis tissue; brain tissue; or testis and brain tissue.

36. The method according to claim 34, wherein the healthy tissue includes testis and brain tissue.

37. The method according to claims 34, wherein the genomic sequences are selected from endogenous viral elements (EVEs), such as endogenous retrovirus (ERV) sequences; novel or unannotated open reading frame (nuOFR) sequences; and genomic sequences that are transcribed as alternatively spliced sequences.

38. The method according to claim 37, wherein the genomic sequences are ERV sequences, in particular human ERV sequences.

39. The method according to claim 34, wherein the amino acid sequences of peptides that will bind to MHC molecules of the patient are amino acid sequences that will
bind both MHC Class I and MHC Class II molecules of the patient;
bind MHC Class I molecules, but not MHC Class II molecules of the patient; or
bind MHC Class II molecules, but not MHC Class I molecules of the patient.

40. The method according to claim 34, wherein the amino acid sequences of the peptides that will bind to MHC molecules of the patient are presented by the MHC molecules on the surface of antigen presenting cells.

41. The method according to claim 34, wherein step A comprises determination of the amino acid sequence from mRNA of the patient's malignant tissue.

42. The method according to claim 34, wherein the selected genomic sequences are identified by determining the expression profile of genomic sequences across a plurality of samples from a plurality of tissues to select those genomic sequences that are expressed in <5% of the plurality of samples.

43. The method according to claim 42, wherein the plurality of samples of a plurality of tissues does not include samples from testis and brain tissue.

44. The method according to claim 34, which is provided as part of a combination treatment of the malignant neoplasm, where the patient also receives a treatment selected from the group consisting of other therapeutic cancer vaccination, chemotherapy, radiotherapy, cytokine therapy, adoptive T-cell therapy, such as CAR-T therapy, targeted antibody therapy, and immune checkpoint inhibitor therapy.

45. The method according to claim 34, wherein, if one or more peptides are administered, the one or more peptides are formulated with an immunological adjuvant.

46. The method according to claim 34, wherein the patient receives a priming immunization and one or more booster immunizations.

47. The method according to claim 44, wherein the other therapeutic cancer vaccination is vaccination that induces immune responses against neoepitopes or neoantigens.

48. The method according to 34, wherein the peptides identified in step C further have been identified in a plurality of cancer patients.

49. A computer or computer system for identifying immunogenic amino acid sequences in a sample of malignant tissue from a patient, said computer or computer system comprising
1) an input component for inputting amino acid sequence or mRNA data;
2) optionally executable code for determining amino acid sequences from mRNA data;
3) a database comprising amino acid sequences of expression products of genomic sequences;
4) executable code for identifying presence—in inputted amino sequences or amino acid sequences encoded by inputted mRNA—of sequences present in the amino acid sequences in the database;
5) executable code that identifies and optionally ranks amino acid sequences identified by the executable code in 4 in accordance with their predicted ability to bind a selection of MHC molecules; and
6) a component for outputting or storing the amino acid sequences identified and/or ranked
wherein said genomic sequences in 3 constitute a subset of all sequences of the genome of a species and where said subset is constituted by sequences, which encode proteinaceous expression products in at most 5% of samples from any healthy tissue type found in the species, where said healthy tissue type optionally does not include testis tissue and/or brain tissue.

50. The computer or computer system according to claim 49, which carries out a method for identifying immunogenic amino acid sequences in a sample of malignant tissue from a patient comprising:
A) determining amino acid sequences of proteinaceous expression products from the malignant tissue,
B) analysing said amino acid sequences to identify therein proteinaceous expression products of selected genomic sequences in the patient's species,
C) identifying, in the proteinaceous expression products, the amino acid sequences, which are those that will bind to MHC molecules of the patient,
where said selected genomic sequences constitute a subset of all sequences of the genome of said species and where said subset is constituted by sequences, which encode proteinaceous expression products in at most 5% of samples from any healthy tissue, where said healthy tissue is a tissue of a type found in the patient, and where said healthy tissue optionally does not include testis tissue and/or brain tissue.
